SEQUENCE LISTING (57 sequences)

SEQ ID NO: 1 - DNA, 40 bp, Artificial Sequence
  Synthetic nucleotide
  misc_feature (27)..(31): n is deoxyinosine
aggatagagaayataaaamatgaacannnnncaacatggc

SEQ ID NO: 2 - DNA, 34 bp, Artificial Sequence
  Synthetic nucleotide
  misc_feature (21)..(25): n is deoxyinosine
ctcacaggaarccaacrtatnnnnnagacgtgga

SEQ ID NO: 3 - DNA, 27 bp, Artificial Sequence
  Synthetic nucleotide
accatrgatgaggctgatcctgatggc

SEQ ID NO: 4 - DNA, 29 bp, Artificial Sequence
  Synthetic nucleotide
acctcatatgatccrtgrtaggcccatgt

SEQ ID NO: 5 - DNA, 33 bp, Artificial Sequence
  Synthetic nucleotide
  misc_feature (20)..(24): n is deoxyinosine
cctctgttgtccaaagggtnnnnngtcagtcat

SEQ ID NO: 6 - DNA, 34 bp, Artificial Sequence
  Synthetic nucleotide
  misc_feature (21)..(25): n is deoxyinosine
catrgctatttgtgtgaccannnnnatracatcc

SEQ ID NO: 7 - DNA, 32 bp, Artificial Sequence
  Synthetic nucleotide
  misc_feature (18)..(22): n is deoxyinosine
accgcgtgtcractgtrnnnnngctgacaaag

SEQ ID NO: 8 - DNA, 35 bp, Artificial Sequence
  Synthetic nucleotide
  misc_feature (22)..(26): n is deoxyinosine
caatatgctgaaacgcgagagnnnnngcgtgtcra

SEQ ID NO: 9 - DNA, 21 bp, Artificial Sequence
  Synthetic nucleotide
tggaatgctgcarggacgmgg

SEQ ID NO: 10 - DNA, 21 bp, Artificial Sequence
  Synthetic nucleotide
tgccacmarggccatgaacag

SEQ ID NO: 11 - DNA, 26 bp, Artificial Sequence
  Synthetic nucleotide
tgctgttggygggattgtyaggaaac

SEQ ID NO: 12 - DNA, 31 bp, Artificial Sequence
  Synthetic nucleotide
  misc_feature (18)..(22): n is deoxyinosine
gcdgtyctgcgtctcctnnnnnagatgttca

SEQ ID NO: 13 - DNA, 33 bp, Artificial Sequence
  Synthetic nucleotide
  misc_feature (20)..(24): n is deoxyinosine
atcrctgttggaatcagcannnnnatcaygcct

SEQ ID NO: 14 - DNA, 36 bp, Artificial Sequence
  Synthetic nucleotide
  misc_feature (22)..(26): n is deoxyinosine
ttcarcatccttccratctctnnnnngaaccctctc

SEQ ID NO: 15 - DNA, 36 bp, Artificial Sequence
  Synthetic nucleotide
  misc_feature (22)..(26): n is deoxyinosine
tgatgggyaagagagagaarannnnnggagagtttg

SEQ ID NO: 16 - DNA, 24 bp, Artificial Sequence
  Synthetic nucleotide
ggtacatgtggttrggagcyaggt

SEQ ID NO: 17 - DNA, 37 bp, Artificial Sequence
  Synthetic nucleotide
  misc_feature (23)..(27): n is deoxyinosine
tcytctgttattcttgtrtcccnnnnngctgtgtcat

SEQ ID NO: 18 - DNA, 34 bp, Artificial Sequence
  Synthetic nucleotide
  misc_feature (21)..(25): n is deoxyinosine
tttcaatatgctgaaacgcgnnnnnaaccgcgta

SEQ ID NO: 19 - DNA, 33 bp, Artificial Sequence
  Synthetic nucleotide
  misc_feature (20)..(24): n is deoxyinosine
tcaatatgctgaaacgcgmnnnnnaccgcgtmt

SEQ ID NO: 20 - DNA, 27 bp, Artificial Sequence
  Synthetic nucleotide
ggttggtgaagagattctcrachggac

SEQ ID NO: 21 - DNA, 26 bp, Artificial Sequence
  Synthetic nucleotide
gggaaaggacccttacggatggtgyt

SEQ ID NO: 22 - DNA, 31 bp, Artificial Sequence
  Synthetic nucleotide
  misc_feature (18)..(22): n is deoxyinosine
gaatccctgctgttggynnnnnggaaagrac

SEQ ID NO: 23 - DNA, 33 bp, Artificial Sequence
  Synthetic nucleotide
  misc_feature (20)..(24): n is deoxyinosine
arcatgcgrcctatctcctnnnnnaatccaatc

SEQ ID NO: 24 - DNA, 34 bp, Artificial Sequence
  Synthetic nucleotide
  misc_feature (20)..(24): n is deoxyinosine
gcctttctcattcgttgtcnnnnngctcctacct

SEQ ID NO: 25 - DNA, 22 bp, Artificial Sequence
  Synthetic nucleotide
cgttgacgctccctacgggtgg

SEQ ID NO: 26 - DNA, 31 bp, Artificial Sequence
  Synthetic nucleotide
  misc_feature (17)..(21): n is deoxyinosine
cccctcgatgcatggcnnnnntttgggcctt

SEQ ID NO: 27 - DNA, 32 bp, Artificial Sequence
  Synthetic nucleotide
  misc_feature (19)..(23): n is deoxyinosine
gcaharagagagggagctnnnnnaacagggaa

SEQ ID NO: 28 - DNA, 36 bp, Artificial Sequence
  Synthetic nucleotide
  misc_feature (22)..(26): n is deoxyinosine
aycaatggaactcagcaaaagnnnnngtggaagayg

SEQ ID NO: 29 - DNA, 27 bp, Artificial Sequence
  Synthetic nucleotide
tcccarccacatgtaccatattgcrcg

SEQ ID NO: 30 - DNA, 27 bp, Artificial Sequence
  Synthetic nucleotide
tccccatcatgttgtaracacacgtrg

SEQ ID NO: 31 - DNA, 34 bp, Artificial Sequence
  Synthetic nucleotide
  misc_feature (21)..(25): n is deoxyinosine
tgaattctctctgctgaaccnnnnntcttcgttc

SEQ ID NO: 32 - DNA, 35 bp, Artificial Sequence
  Synthetic nucleotide
  misc_feature (21)..(25): n is deoxyinosine
ctctactgaaccagtgatctnnnnncatgaaacca

SEQ ID NO: 33 - DNA, 33 bp, Artificial Sequence
  Synthetic nucleotide
  misc_feature (20)..(24): n is deoxyinosine
gtccttctccytccactccnnnnngtgaattct

SEQ ID NO: 34 - DNA, 32 bp, Artificial Sequence
  Synthetic nucleotide
  misc_feature (19)..(23): n is deoxyinosine
accctcatggccatagaynnnnntgarttgtg

SEQ ID NO: 35 - DNA, 33 bp, Artificial Sequence
  Synthetic nucleotide
  misc_feature (19)..(23): n is deoxyinosine
gccatdgaccttggtgaannnnntgaagayaca

SEQ ID NO: 36 - DNA, 33 bp, Artificial Sequence
  Synthetic nucleotide
  misc_feature (19)..(23): n is deoxyinosine
ctcaggcagaatgarccannnnncatagaytgt

SEQ ID NO: 37 - DNA, 33 bp, Artificial Sequence
  Synthetic nucleotide
  misc_feature (20)..(24): n is deoxyinosine
ttggtgarttgtgtgaagannnnntcacgtaya

SEQ ID NO: 38 - DNA, 24 bp, Artificial Sequence
  Synthetic nucleotide
tggagacacgaactgaaacatgga

SEQ ID NO: 39 - DNA, 27 bp, Artificial Sequence
  Synthetic nucleotide
tggaaacgagaactgaaacatggatgt

SEQ ID NO: 40 - DNA, 24 bp, Artificial Sequence
  Synthetic nucleotide
tggatgtcrtcagaaggggcytgg

SEQ ID NO: 41 - DNA, 33 bp, Artificial Sequence
  Synthetic nucleotide
  misc_feature (20)..(24): n is deoxyinosine
gccaggattgctgccattannnnnaagcctgga

SEQ ID NO: 42 - DNA, 34 bp, Artificial Sequence
  Synthetic nucleotide
  misc_feature (20)..(24): n is deoxyinosine
ctggatgtctcargayccannnnncaattctctg

SEQ ID NO: 43 - DNA, 35 bp, Artificial Sequence
  Synthetic nucleotide
  misc_feature (21)..(25): n is deoxyinosine
cctatggtgtatgccargatnnnnnccattatggt

SEQ ID NO: 44 - DNA, 33 bp, Artificial Sequence
  Synthetic nucleotide
  misc_feature (19)..(23): n is deoxyinosine
ayggaggaatgcttgtgannnnnccactytcac

SEQ ID NO: 45 - DNA, 26 bp, Artificial Sequence
  Synthetic nucleotide
tccatrttgggtgtttctggttcygc

SEQ ID NO: 46 - DNA, 34 bp, Artificial Sequence
  Synthetic nucleotide
  misc_feature (21)..(25): n is deoxyinosine
actccrtttatcatggaggannnnnagcctgtrg

SEQ ID NO: 47 - DNA, 31 bp, Artificial Sequence
  Synthetic nucleotide
  misc_feature (18)..(22): n is deoxyinosine
cccttacggatggtgytnnnnntcatyacgt

SEQ ID NO: 48 - DNA, 33 bp, Artificial Sequence
  Synthetic nucleotide
  misc_feature (20)..(24): n is deoxyinosine
agattctcracyggactttnnnnngggaaagga

SEQ ID NO: 49 - DNA, 22 bp, Artificial Sequence
  Synthetic nucleotide
tcccaccaacagcagggattct

SEQ ID NO: 50 - DNA, 25 bp, Artificial Sequence
  Synthetic nucleotide
tgcgrcctatctccttcctraatcc

SEQ ID NO: 51 - DNA, 32 bp, Artificial Sequence
  Synthetic nucleotide
  misc_feature (19)..(23): n is deoxyinosine
gccattacggtgggaaycnnnnncarcaatgt

SEQ ID NO: 52 - DNA, 33 bp, Artificial Sequence
  Synthetic nucleotide
  misc_feature (20)..(24): n is deoxyinosine
gaatcaagcacarcartgtnnnnnttgaccttt

SEQ ID NO: 53 - DNA, 34 bp, Artificial Sequence
  Synthetic nucleotide
  misc_feature (20)..(24): n is deoxyinosine
acargtgaaacgccattacnnnnngaatcaarca

SEQ ID NO: 54 - DNA, 34 bp, Artificial Sequence
  Synthetic nucleotide
  misc_feature (21)..(25): n is deoxyinosine
gttgtcttraacaagagaggnnnnnccctttcrt

SEQ ID NO: 55 - DNA, 32 bp, Artificial Sequence
  Synthetic nucleotide
  misc_feature (18)..(22): n is deoxyinosine
gggtggactgtggagagnnnnngcactgctaa

SEQ ID NO: 56 - DNA, 22 bp, Artificial Sequence
  Synthetic nucleotide
ccgtatggccaacaactggcgc

SEQ ID NO: 57 - DNA, 36 bp, Artificial Sequence
  Synthetic nucleotide
  misc_feature (22)..(26): n is deoxyinosine
gctaacgcatctaaggtatggnnnnncgagaaagga

(19) United States
(12) Patent Application Publication
(10) Pub. No.: US 2022/0372571 A1
(43) Pub. Date: Nov. 24, 2022

(54) METHODS FOR PREPARING AN OPTIMAL COMBINATION OF OLIGONUCLEOTIDE SETS

(71) Applicant: SEEGENE, INC., Seoul (KR)

(72) Inventor: Je-Hwan PARK, Seoul (KR)

(73) Assignee: SEEGENE, INC., Seoul (KR)

(21) Appl. No.: 17/772,197

(22) PCT Filed: Nov. 26, 2020
(86) PCT No.: PCT/KR2020/016965

§ 371 Date: Apr. 27, 2022

(30) Foreign Application Priority Data
Nov. 29, 2019 (KR) 10-2019-0157540

(51) Int. Cl. C12Q 1/6876; C12Q 1/6869; G16B 30/10; G16B 25/20

(52) U.S. Cl. CPC C12Q 1/6876; C12Q 1/6869; G16B 30/10; G16B 25/20

(57) ABSTRACT

The present invention relates to technologies for preparing an optimal combination of oligonucleotide sets used to simultaneously detect a plurality of target nucleic acid molecules. Unlike a conventional method of checking whether a dimer is formed in all candidate combinations of oligonucleotide sets, the present invention is capable of providing a combination of oligonucleotide sets used to detect a plurality of target nucleic acid molecules with speed and accuracy, by replacing only an oligonucleotide set with dimer formation in a first reference combination of oligonucleotide sets to provide, as a new reference combination, a combination with a reduction in dimer formation compared with the first reference combination, and replacing only an oligonucleotide set with dimer formation in the new reference combination to provide a combination with all dimers removed.

### CROSS-REFERENCE TO RELATED APPLICATIONS

This application claims priority from Korean Patent Application No. 2019-0157540, filed on Nov. 29, 2019 in the Korean Intellectual Property Office, the disclosure of which is incorporated herein by reference.

### BACKGROUND OF THE INVENTION

### Field of the Invention

The present invention relates to technologies for preparing an optimal combination of oligonucleotide sets used to simultaneously detect a plurality of target nucleic acid molecules.

### Description of the Related Art

The polymerase chain reaction (PCR), which is a nucleic acid amplification method, involves repeated cycles of denaturation of double-stranded DNA, oligonucleotide primer annealing to the DNA template, and primer extension by a DNA polymerase (Mullis et al., U.S. Pat. Nos. 4,683,195, 4,683,202, and 4,800,159; and Saiki et al., (1985) Science 230, 1350-1354).

PCR-based techniques have been widely used for amplification of target DNA sequences as well as scientific applications or methods in the fields of biological and medical research, such as reverse transcriptase PCR (RT-PCR), differential display PCR (DD-PCR), cloning of known or unknown genes by PCR, rapid amplification of cDNA ends (RACE), arbitrary priming PCR (AP-PCR), multiplex PCR, SNP genome typing, and PCR-based genomic analysis (McPherson and Moller, (2000) PCR. BIOS Scientific Publishers, Springer-Verlag New York Berlin Heidelberg, N.Y.).

Out of the PCR-based techniques, multiplex PCR means the simultaneous amplification and detection of multiple regions of one target nucleic acid molecule or a plurality of target nucleic acid molecules by using a combination of a plurality of oligonucleotide sets (forward and reverse primers and probes) in one tube.

To provide a combination of a plurality of oligonucleotide sets for multiplex PCR, an oligonucleotide set having a performance capable of detecting a plurality of nucleic acid sequences of a particular target nucleic acid molecule with maximum coverage needs to be designed, and a pool of oligonucleotide sets including such oligonucleotide sets needs to be provided. The oligonucleotides (primers and probes) included in the oligonucleotide sets are designed in consideration of Tm value, nucleotide length, and the like, and the oligonucleotide sets are provided in consideration of amplicon sizes and dimer formation.

To run multiplex PCR by using such oligonucleotide sets, it is important that there is no interference among a plurality of oligonucleotide sets, and a representative phenomenon of such interference is dimer formation. Although having excellent characteristics, oligonucleotide sets cannot be provided as a combination of oligonucleotide sets when a dimer is formed between oligonucleotide sets designed to detect different target nucleic acid molecules.

In the checking of whether a dimer is formed between oligonucleotide sets in candidate combinations of oligonucleotide sets for multiplex PCR, when the size of a pool of oligonucleotide sets is not large, an optimal combination of oligonucleotide sets is able to be provided by checking for whether a dimer is formed in all the candidate combinations. However, when the size of a pool of oligonucleotide sets is large and the numbers of oligonucleotides included in the oligonucleotide sets are large, not only does it take a long time to check whether a dimer is formed in all the candidate combinations, but it is also not possible to check whether a dimer is formed in all the candidate combinations, and thus, an optimal combination of oligonucleotide sets cannot be provided.

Therefore, the present inventors have recognized the need to develop a technique capable of efficiently providing an optimal combination of oligonucleotide sets for multiplex PCR.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches to develop a method being capable of efficiently combining a plurality of oligonucleotide sets (for example, primer pairs and probes) used to amplify and detect a plurality of target nucleic acid molecules. As a result, unlike a conventional method of checking whether a dimer is formed in all candidate combinations of oligonucleotide sets, the present inventors have found that a combination of oligonucleotide sets used to detect a plurality of target nucleic acid molecules can be provided with speed and accuracy, by replacing only an oligonucleotide set with dimer formation in a first reference combination of oligonucleotide sets to provide, as a new reference combination, a combination with a reduction in dimer formation compared with the first reference combination, and replacing only an oligonucleotide set with dimer formation in the new reference combination to provide a combination with all dimers removed.

Accordingly, it is an object of this invention to provide a method for preparing an optimal combination of oligonucleotide sets used to simultaneously detect a plurality of target nucleic acid molecules.

It is another object of this invention to provide a computer readable storage medium containing instructions to configure a processor to perform a method for preparing an optimal combination of oligonucleotide sets used to simultaneously detect a plurality of target nucleic acid molecules.

Other objects and advantages of the present invention will become apparent from the detailed description to follow taken in conjugation with the appended claims and drawings.

### BRIEF DESCRIPTION OF THE DRAWINGS

FIG. 1 is a flow chart of processes for performing the method of the present invention according to an embodiment of the present invention.

FIG. 2 shows pools of oligonucleotide sets (OSs) used to detect target nucleic acid molecules of eight organisms or analytes.

FIGS. 3 and 4 show a procedure of providing an optimal combination of oligonucleotide sets used to simultaneously detect target nucleic acid molecules of five organisms according to an embodiment of the present invention.

### DETAILED DESCRIPTION OF THIS INVENTION

In one aspect of the present invention, there is provided a method for preparing an optimal combination of oligonucleotide sets used to simultaneously detect a plurality of target nucleic acid molecules, comprising:

(a) providing a pool of oligonucleotide sets used to detect each of the plurality of target nucleic acid molecules for each of the plurality of target nucleic acid molecules; wherein the oligonucleotide sets each comprises one or more oligonucleotides,

(b) providing, as a first reference combination, a combination of oligonucleotide sets combined from the pool of oligonucleotide sets provided for each of the plurality of target nucleic acid molecules, and checking whether a dimer is formed between oligonucleotide sets of the combination;

(c) replacing an oligonucleotide set with dimer formation in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether dimer formation is reduced compared with the first reference combination;

(d) in the combination checking for whether the dimer formation is reduced, providing, as a second reference combination, a combination of oligonucleotide sets with a reduction in dimer formation compared with the first reference combination;

(e) replacing an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and checking whether a dimer is formed between oligonucleotide sets of the combination; and

(f) in the combination checking for whether the dimer is formed, providing a combination of oligonucleotide sets with no dimer formation; wherein the combination of oligonucleotide sets with no dimer formation is used to simultaneously detect the plurality of target nucleic acid molecules.

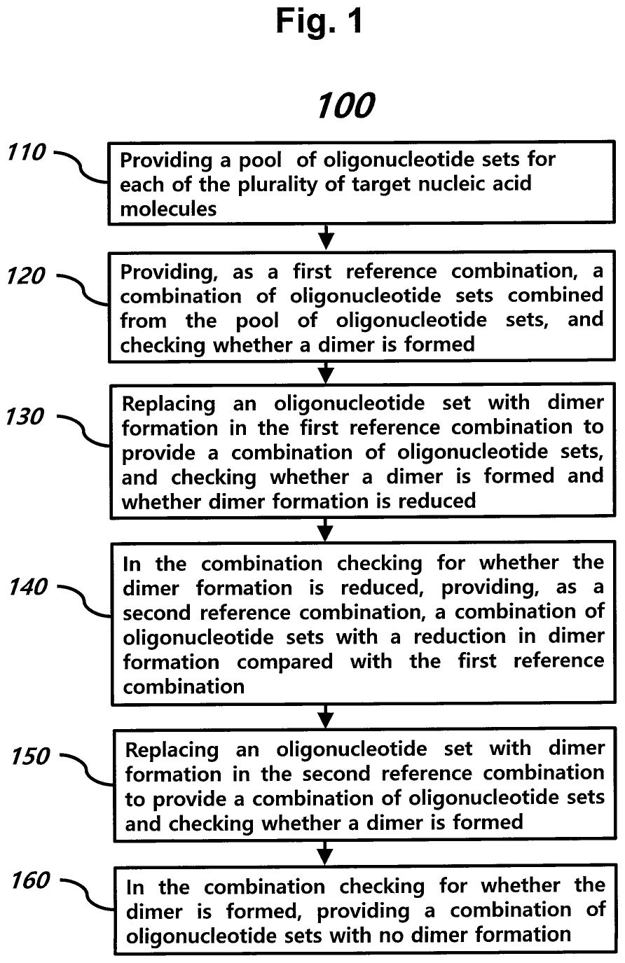

FIG. 1 is a flow chart showing steps for implementing a method of the present invention according to an embodiment of the present invention. The method of the present invention will be described with reference to FIG. 1 as follows.

### Step (a): Providing a Pool of Oligonucleotide Sets for Each of the Plurality of Target Nucleic Acid Molecules (110)

First, the method of the present invention provides a pool of oligonucleotide sets used to detect each of the plurality of target nucleic acid molecules for each of the plurality of target nucleic acid molecules. The oligonucleotide sets each comprises one or more oligonucleotides.

The term used herein “target nucleic acid molecule”, “target molecule”, or “target nucleic acid” refers to a nucleotide molecule in an organism to be detected. A target nucleic acid molecule is generally given a particular name, and includes the whole genome and all nucleotide molecules constituting the genome (e.g., genes, pseudogenes, non-coding sequence molecules, untranslated region and some regions of the genome). A target nucleic acid molecule includes, for example, nucleic acids of the organism.

As used herein, the term “target nucleic acid sequence” or “target sequence” refers to a particular target nucleic acid sequence representing a target nucleic acid molecule.

As used herein, the term “organism” refers to an organism that belongs to one genus, species, subspecies, subtype, genotype, serotype, strain, isolate, or cultivar. Examples of the organism include prokaryotic cells (e.g., Mycoplasma pneumoniae, Chlamydophila pneumoniae, Legionella pneumophila, Haemophilus influenzae, Streptococcus pneumoniae, Bordetella pertussis, Bordetella parapertussis, Neisseria meningitidis, Listeria monocytogenes, Streptococcus agalactiae, Campylobacter, Clostridium difficile, Clostridium perfringens, Salmonella, Escherichia coli, Shigella, Vibrio, Yersinia enterocolitica, Aeromonas, Chlamydia trachomatis, Neisseria gonorrhoeae, Trichomonas vaginalis, Mycoplasma hominis, Mycoplasma genitalium, Ureaplasma urealyticum, Ureaplasma parvum, Mycobacterium tuberculosis), eukaryotic cells (e.g., protozoa and parasites, fungi, yeasts, higher plants, lower animals, and higher animals including mammals and humans), viruses, or viroids. Examples of the parasites of the prokaryotic cells include Giardia lamblia, Entamoeba histolytica, Cryptosporidium, Blastocystis hominis, Dientamoeba fragilis, and Cyclospora cayetanensis. Examples of the viruses include: influenza A virus (Flu A), influenza B virus (Flu B), respiratory syncytial virus A (RSV A), respiratory syncytial virus B (RSV B), parainfluenza virus 1 (PIV 1), parainfluenza virus 2 (PIV 2), parainfluenza virus 3 (PIV 3), parainfluenza virus 4 (PIV 4), metapneumovirus (MPV), Human enterovirus (HEV), human bocavirus (HBoV), human rhinovirus (HRV), coronavirus, and adenovirus, which cause respiratory diseases; and norovirus, rotavirus, adenovirus, astrovirus, and sapovirus, which cause gastrointestinal diseases. Examples of the viruses also include human papillomavirus (HPV), middle east respiratory syndrome-related coronavirus (MERS-CoV), dengue virus, herpes simplex virus (HSV), human herpes virus (HHV), Epstein-Barr virus (EMV), varicella zoster virus (VZV), cytomegalovirus (CMV), HIV, hepatitis virus, and poliovirus.

According to an embodiment of the present invention, the plurality of target nucleic acid molecules are target nucleic acid molecules of one organism or a plurality of organisms. In the present invention, the plurality of organisms are at least two organisms, for example, organisms selected from 2 to 20 organisms, and the plurality of organisms may include all different organisms, some same organisms, or all same organisms.

As used herein, the term “a pool of oligonucleotide sets” refers to a group including oligonucleotide sets used to detect target nucleic acid molecules to be detected.

In the present invention, the oligonucleotide sets each comprises one or more oligonucleotides, and particularly, the oligonucleotide comprises a primer and/or a probe, and more particularly, a primer pair and/or a probe. Most particularly, the oligonucleotide set may contain one primer pair and one probe, or two or more primer pairs and one or more probe.

According to another embodiment, the oligonucleotide set is used to detect one or more target nucleic acid molecules of the same organism. An oligonucleotide set included in a pool of oligonucleotide sets provided for each of a plurality of target nucleic acid molecules may be used to detect one target nucleic acid molecule or two or more target nucleic acid molecules of the same organism.

As used herein, the term “oligonucleotide” refers to a linear oligomer of natural or modified monomers or linkages. The oligonucleotide includes deoxyribonucleotides and ribonucleotides, can specifically hybridize with a target nucleotide sequence, and is naturally present or artificially synthesized. The oligonucleotide is especially a single chain for maximal efficiency in hybridization. Particularly, the oligonucleotide is an oligodeoxyribonucleotide. The oligonucleotide of the present invention may include naturally occurring dNMPs (i.e., dAMP, dGMP, dCMP and dTMP), or nucleotide analogs or derivatives. The oligonucleotide may also include a ribonucleotide. For example, the oligonucleotide of the present invention may include backbone-modified nucleotides, such as peptide nucleic acid (PNA) (M. Egholm et al., Nature, 365:566-568 (1993)), locked nucleic acid (LNA) (WO1999/014226), bridged nucleic acid (BNA) (WO2005/021570), phosphorothioate DNA, phosphorodithioate DNA, phosphoramidate DNA, amide-linked DNA, MMI-linked DNA, 2′-O-methyl RNA, alpha-DNA and methylphosphonate DNA, sugar-modified nucleotides, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and base-modified nucleotides, such as C-5 substituted pyrimidine (substituent including fluoro-, bromo-, chloro-, iodo-, methyl-, ethyl-, vinyl-, formyl-, ethynyl-, propynyl-, alkynyl-, thiazolyl-, imidazolyl-, and pyridyl-), 7-deazapurines with C-7 substituent (substituent including fluoro-, bromo-, chloro-, iodo-, methyl-, ethyl-, vinyl-, formyl-, alkynyl-, alkenyl-, thiazolyl-, imidazolyl-, and pyridyl-), inosine, and diaminopurine. Especially, the term “oligonucleotide” used herein is a single strand composed of deoxyribonucleotides. The term “oligonucleotide” includes oligonucleotides that hybridize with cleavage fragments occurring depending on the target nucleic acid sequence. Particularly, the oligonucleotide comprises a primer and/or a probe.

As used herein, the term “primer” refers to an oligonucleotide, which is capable of acting as a point of initiation of synthesis when placed under conditions in which synthesis of primer extension product which is complementary to a nucleic acid strand (a template) is induced, i.e., in the presence of nucleotides and an agent for polymerization, such as DNA polymerase, and at a suitable temperature and pH. The primer should be long enough to prime the synthesis of the extension product in the presence of an agent for polymerization. The suitable length of the primer depends on a plurality of factors, such as temperature, a field of application, and a primer source.

The primer may have a length of, for example, 10-100 nucleotides, 10-80 nucleotides, 10-50 nucleotides, 10-40 nucleotides, 10-30 nucleotides, 15-100 nucleotides, 15-80 nucleotides, 15-50 nucleotides, 15-40 nucleotides, 15-30 nucleotides, 20-100 nucleotides, 20-80 nucleotides, 20-50 nucleotides, 20-40 nucleotides, or 20-30 nucleotides. When the primer is a DPO primer developed by the present applicant (see U.S. Pat. No. 8,092,997), the descriptions of the length of the DPO primer disclosed in the patent document are incorporated herein by reference.

As used herein, the term “probe” refers to a single-stranded nucleic acid molecule containing a portion or portions that are complementary to a target nucleic acid sequence. The probe may also contain a label capable of generating a signal for target detection.

The probe may have a length of, for example, 10-100 nucleotides, 10-80 nucleotides, 10-50 nucleotides, 10-40 nucleotides, 10-30 nucleotides, 15-100 nucleotides, 15-80 nucleotides, 15-50 nucleotides, 15-40 nucleotides, 15-30 nucleotides, 20-100 nucleotides, 20-80 nucleotides, 20-50 nucleotides, 20-40 nucleotides, or 20-30 nucleotides in length. When the probe is a tagging probe, descriptions of the length are applied to a targeting portion of the tagging probe. The tagging portion of the tagging probe may have a length of, for example, 7-48 nucleotides, 7-40 nucleotides, 7-30 nucleotides, 7-20 nucleotides, 10-48 nucleotides, 10-40 nucleotides, 10-30 nucleotides, 10-20 nucleotides, 12-48 nucleotides, 12-40 nucleotides, 12-30 nucleotides, or 12-20 nucleotides, but is not limited thereto.

The oligonucleotide may have a conventional primer and probe structure consisting of sequences that are hybridized with a target nucleic acid sequence. Alternatively, the oligonucleotides may have a unique structure through structural modification thereof. For example, the oligonucleotides may have structures of Scorpion primer, Molecular beacon probe, Sunrise primer, HyBeacon probe, tagging probe, DPO primer or probe (WO 2006/095981), and PTO probe (WO 2012/096523).

The oligonucleotide may be a modified oligonucleotide, such as a degenerate base-containing oligonucleotide and/or a universal base-containing oligonucleotide, in which degenerate bases and/or universal bases are introduced into a conventional primer or probe. As used herein, the terms “conventional primer”, “conventional probe”, and “conventional oligonucleotide” refer to a general primer, a probe, and an oligonucleotide, into which a degenerate base or non-natural base is not introduced. According to an embodiment of the present invention, the degenerate base-containing oligonucleotide or universal base-containing oligonucleotide is an oligonucleotide of which at least 50%, at least 60%, at least 70%, at least 80%, at least 90%, or at least 95% is not modified. According to an embodiment, the number of degenerate bases or universal bases introduced into the conventional oligonucleotide is particularly 7 or less, 5 or less, 4 or less, 3 or less, or 2 or less. In addition, the use ratio of degenerate bases and/or universal bases introduced into the conventional oligonucleotide is particularly 25% or less, 20% or less, 18% or less, 16% or less, 14% or less, 12% or less, 10% or less, 8% or less, or 6% or less. The use ratio of degenerate bases or universal bases represents a ratio of degenerate bases or universal bases over all the nucleotides of the oligonucleotide into which degenerate bases or universal bases are introduced. The degenerate bases include various degenerate bases known in the art as follows: R: A or G; Y: C or T; S: G or C; W: A or T; K: G or T; M: A or C; B: C or G or T; D: A or G or T; H: A or C or T; V: A or C or G; N: A or C or G or T. The universal bases include various universal bases known in the art as follows: deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitropyrrole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof. More particularly, the universal base is deoxyinosine, inosine, or a combination thereof.

According to another embodiment of the present invention, the oligonucleotide sets in step (a) are ranked based on predetermined sorting criteria. The ranks are given by serial numbers to oligonucleotide sets.

According to still more another embodiment, the ranking of the oligonucleotide sets in step (a) is carried out by ranking based on at least one of the following predetermined sorting criteria:

(i) the total sum of the number of oligonucleotides contained in an oligonucleotide set; the smaller the total sum, the higher the priority,

(ii) the total sum of the number of a degenerate base and/or universal base introduced into an oligonucleotide contained in an oligonucleotide set; the smaller the total sum, the higher the priority,

(iii) a target-coverage of an oligonucleotide set for a plurality of target nucleic acid sequences of a target nucleic acid molecule; the larger the target-coverage, the higher the priority, and

(iv) the total sum of the number of oligonucleotide patterns generated by a degenerate base introduced into an oligonucleotide contained in an oligonucleotide set; the smaller the total sum, the higher the priority.

The oligonucleotide sets included in a pool of oligonucleotide sets for each of a plurality of target nucleic acid molecules are ranked based on at least one (particularly, sorting criterion (i)), specifically at least two, more specifically at least three, and still more specifically at least four sorting criteria.

According to another embodiment of the present invention, the at least two sorting criteria have a difference in criticality, and the ranks of oligonucleotide sets included in a pool of oligonucleotide sets are assigned by ranks according to the at least two sorting criteria considering the criticality. For example, when the number of first-rank oligonucleotide sets is plural based on the sorting criterion with the highest criticality, an oligonucleotide set having the highest rank is selected by comparison of ranks based on the next priority sorting criterion.

According to another embodiment, when different weights are assigned to the sorting criteria and scores are assigned to values (or value ranges) in each of the sorting criteria, the total score of each of the oligonucleotide sets can be obtained. Considering the calculated total scores, the ranks of the oligonucleotide sets included in a pool of oligonucleotide sets can be given.

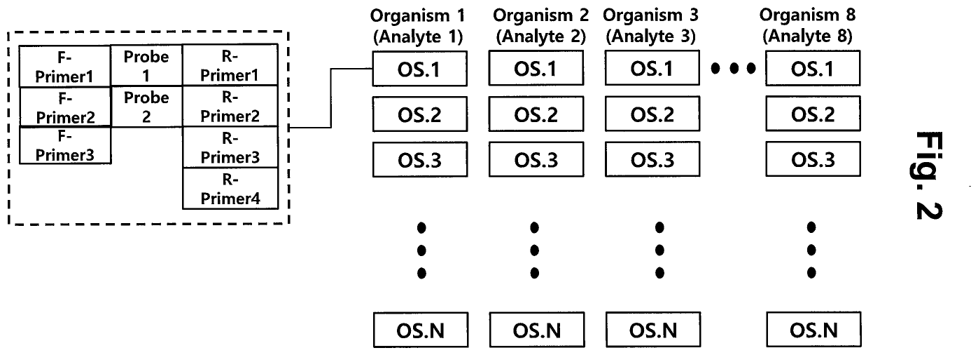

FIG. 2 shows pools of oligonucleotide sets (OSs) used to detect target nucleic acid molecules of eight organisms (or analytes). For example, it is shown that oligonucleotide set 1 (OS.1) of organism 1 (analyte 1) is composed of three forward primers, two probes, and four reverse primers, and a pool of oligonucleotide sets used to detect each of target nucleic acid molecules of eight organisms includes oligonucleotide sets with ranks of 1 to N.

Step (b): Providing, as a First Reference Combination, a Combination of Oligonucleotide Sets Combined from the Pool of Oligonucleotide Sets, and Checking Whether a Dimer is Formed (120)

Then, the method of the present invention provides, as a first reference combination, a combination of oligonucleotide sets combined from the pool of oligonucleotide sets provided for each of the plurality of target nucleic acid molecules, and checks whether a dimer is formed between oligonucleotide sets of the combination.

According to the present invention, the first reference combination is provided as a first candidate of an optimal combination of oligonucleotide sets for use in the simultaneous detection of a plurality of target nucleic acid molecules. When no dimer formation is confirmed by checking whether a dimer is formed between oligonucleotide sets of the first reference combination, the oligonucleotide sets of the first reference combination can be used to simultaneously detect a plurality of target nucleic acid molecules. However, when at least one dimer is formed between oligonucleotide sets of the first reference combination, oligonucleotide sets with dimer formation in the first reference combination are replaced according to the method to be described below, so that a combination of oligonucleotide sets with no dimer formation can be provided.

The first reference combination is a combination of oligonucleotide sets randomly selected from a pool of oligonucleotide sets provided for each of the plurality of target nucleic acid molecules, and particularly, the first reference combination has a predetermined rank sum, and more particularly the minimum rank sum.

As used herein, the term “rank sum” refers to a sum of ranks given to respective oligonucleotide sets included in a combination of the oligonucleotide sets. Particularly, when in FIG. 2, a combination with the minimum rank sum is provided as a first reference combination, a combination composed of oligonucleotide sets with rank 1 in eight organisms, that is, the combination of [1, 1, 1, 1, 1, 1, 1, 1] indicating a rank sum of 8 may be provided as the first reference combination.

According to the present invention, the first reference combination is provided, and then it is checked whether a dimer is formed between oligonucleotide sets of the combination.

According to another embodiment of the present invention, the checking of whether the dimer is formed is carried out by confirming whether one or more of the following criteria are satisfied: (i) the proportion of total nucleotides forming Watson-Crick base pairs between oligonucleotides is at least a predetermined value (a predetermined value or more) (particularly, 20% 30%, 40%, 50%, 60%, or 70%); and (ii) the proportion of consecutive nucleotides forming Watson-Crick base pairs between oligonucleotides is at least a predetermined value (a predetermined value or more) (particularly, 10%, 15%, 20%, 25%, 30%, or 35%).

For example, the checking of whether a dimer is formed between oligonucleotide 1 contained in an oligonucleotide set of organism 1 and oligonucleotide 2 contained in an oligonucleotide set of organism 2 is determined depending on the proportion of nucleotides involved in the formation of Watson-Crick base pairs between oligonucleotide 1 in the direction of 5′ to 3′ and oligonucleotide 2 in the direction of 3′ to 5′. In a case where both of the oligonucleotide 1 and oligonucleotide 2 contain 30 bases and the numbers of inconsecutive and consecutive nucleotides involved in the formation of Watson-Crick base pairs are 6 bases and 2 bases, respectively, there is no dimer formation between oligonucleotide 1 and oligonucleotide 2 according to the criterion (ii) (a predetermined value of 10% or more) but there is dimer formation according to the criterion (i) (a predetermined value of 20% or more).

According to another embodiment, the dimer formation is expressed as a dimer link (D-link) and/or a dimer level (D-level), the dimer link represents one or more dimer pairs formed between two oligonucleotide sets of the oligonucleotide sets of the combination and the one or more dimer pairs are considered to be one dimer link, and the dimer level represents the minimum number of oligonucleotide sets that need to be replaced in order to remove all dimer links formed between oligonucleotide sets of the combination or the minimum number of times of replacement necessary for removing all dimer links formed between oligonucleotide sets of the combination.

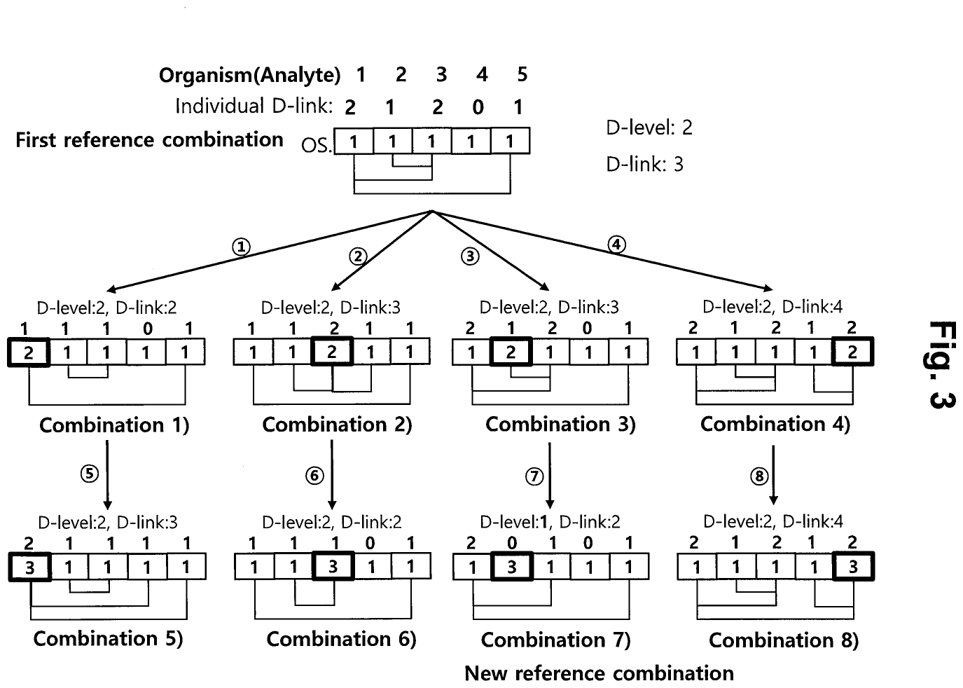

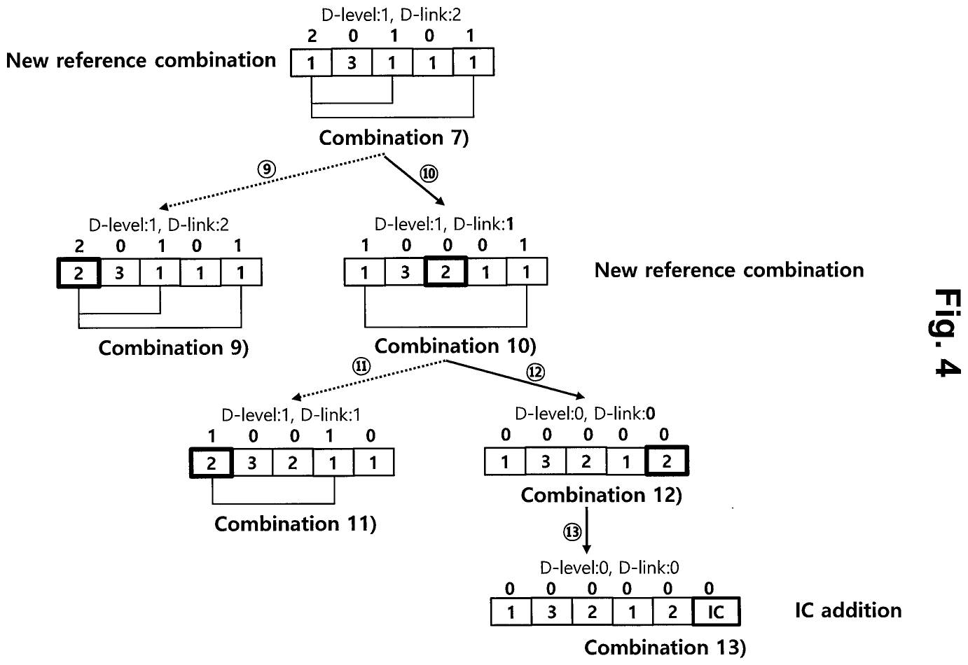

FIGS. 3 and 4 show a procedure of providing an optimal combination of oligonucleotide sets used to simultaneously detect target nucleic acid molecules of five organisms according to an embodiment of the present invention.

In FIG. 3, a combination of oligonucleotide sets with the minimum rank sum, that is, the combination of [1, 1, 1, 1, 1] is provided as the first reference combination, and it is checked whether a dimer is formed between oligonucleotide sets of the combination. As a result, it is confirmed that dimers are formed between oligonucleotide sets of organism 1 and organism 3, oligonucleotide sets of organism 1 and organism 5, and oligonucleotide sets of organism 2 and organism 3. As can be confirmed in FIG. 2, an oligonucleotide set contains at least three oligonucleotides, and thus for example, the number of dimer pairs formed between the oligonucleotide sets of organism 1 and organism 3 may be at least one, but at least one dimer pair is considered to be one dimer link.

As used herein, “the number of dimer links” represents the total number of dimer links in a combination of oligonucleotide sets, and “the number of individual dimer links” represents the number of dimer links involved in each oligonucleotide set in a combination of oligonucleotide sets.

In the first reference combination of FIG. 3, the number of dimer links (D-link) indicating dimer formation is 3, and the number of individual dimer links involved in each oligonucleotide sets of the first reference combination is 2 for the oligonucleotide set of organism 1, 1 for organism 2, 2 for organism 3, 0 for organism 4, and 1 for organism 5.

In the first reference combination of FIG. 3, when the oligonucleotide set of organism 1 is replaced with another oligonucleotide set, the dimer links may be removed between the oligonucleotide sets of organism 1 and organism 3, and the oligonucleotide sets of organism 1 and organism 5, and when the oligonucleotide set of organism 2 or organism 3 is replaced with another oligonucleotide set, the dimer link may be removed between the oligonucleotide sets of organism 2 and organism 3. Therefore, the minimum number of oligonucleotide sets that need to be replaced in order to remove all the dimer links in the first reference combination or the minimum number of replacements necessary for removing all the dimer links in the first reference combination is 2. Accordingly, in the first reference combination of FIG. 3, the dimer level (D-level) indicating whether a dimer is formed is 2. If there is no dimer formation in the first reference combination of FIG. 3, the number of dimer links, the number of individual dimer links, and the dimer level are all 0.

The description of “checking whether a dimer is formed” in the present step is applied to the steps to be described later in the same manner.

Step (c): Replacing an Oligonucleotide Set with Dimer Formation in the First Reference Combination to Provide a Combination of Oligonucleotide Sets, and Checking Whether a Dimer is Formed and Whether Dimer Formation is Reduced (130)

Then, the method of the present invention replaces an oligonucleotide set with dimer formation in the first reference combination with another oligonucleotide set belonging to the same pool of oligonucleotide sets, thereby providing a combination of oligonucleotide sets, which is different from the first reference combination only in the replaced another oligonucleotide set, and checks whether a dimer is formed between oligonucleotide sets of the combination and whether dimer formation is reduced compared with the first reference combination.

One of the features of the present invention is that all the oligonucleotide sets in the first reference combination are not replaced with other oligonucleotide sets, but only oligonucleotide sets with dimer formation are replaced with other oligonucleotide sets, thereby decreasing the number of candidate combinations of oligonucleotide sets checking whether a dimer is formed.

The combination of oligonucleotide sets in step (c) is provided by replacing only an oligonucleotide set with dimer formation in the first reference combination with another oligonucleotide set belonging to the same pool of oligonucleotide sets. According to a specific embodiment, the another oligonucleotide set is a next-rank oligonucleotide set.

According to another embodiment of the present invention, the oligonucleotide set with dimer formation is an oligonucleotide set with a dimer link.

The replacement is carried out by replacing only one oligonucleotide set at one time point. Therefore, the combination of oligonucleotide sets provided in step (c) of the present invention is different from the first reference combination only in the replaced another oligonucleotide set.

After an oligonucleotide set with dimer formation is replaced to provide a combination of oligonucleotide sets, and then it is checked whether a dimer is formed between oligonucleotide sets of the combination. As a result, when no dimer is formed, the combination can be used to simultaneously detect the plurality of target nucleic acid molecules. However, when a dimer is still formed, it is checked whether dimer formation is reduced compared with the first reference combination.

Since the contents of the checking of whether a dimer is formed and the dimer formation are the same as those of step (b), the descriptions thereof are omitted to avoid undue redundancy leading to the complexity of this specification.

The checking of whether dimer formation is reduced compared with the first reference combination is carried out by, specifically, checking whether the number of dimer links and/or the dimer level is reduced.

In the present step, (i) while each combination of oligonucleotide sets from the first reference combination is provided, it may be checked whether a dimer is formed and whether dimer formation is reduced in the combination; or (ii) after all the combinations of oligonucleotide sets from the first reference combination are provided, it may be checked whether a dimer is formed and whether dimer formation is reduced in all the combinations. Particularly, the present step is performed according to option (i) above.

According to another embodiment, the replacement in step (c) is carried out in the order from any one oligonucleotide set to any other oligonucleotide set of the oligonucleotide sets with dimer formation in the first reference combination. More specifically, the order from any one oligonucleotide set to any other oligonucleotide set is (i) an order from an oligonucleotide set with the largest number of individual dimer links to an oligonucleotide set with the smallest number of individual dimer links, and (ii) when the number of individual dimer links is the same, an order from an oligonucleotide set located in the front of a combination to an oligonucleotide set located in the back or an order from an oligonucleotide set belonging to a pool of oligonucleotide sets including the largest number of oligonucleotide sets to an oligonucleotide set belonging to a pool of oligonucleotide sets including the smallest number of oligonucleotide sets.

Referring to FIG. 3, as described above, in the first reference combination, the number of dimer links (D-links) is 3 and the dimer level (D-level) is 2, and the numbers of individual dimer links of oligonucleotide sets for organisms 1 to 5 are 2, 1, 2, 0, and 1, respectively. In an order from an oligonucleotide set with the largest number of individual dimer links in the first reference combination, or in an order from an oligonucleotide set located in the front of the first reference combination when the number of individual dimer links is the same, the oligonucleotide set with a dimer link is replaced with the next-rank oligonucleotide set.

First, the first-rank oligonucleotide set of organism 1 is replaced with the second-rank oligonucleotide belonging to the same pool of oligonucleotide sets to thereby provide combination 1) of [2, 1, 1, 1, 1], and then it is checked whether a dimer is formed (the number of dimer links being 2 and the dimer level being 2) in the combination 1) and whether dimer formation is reduced compared with the first reference combination. In addition, combinations are provided by respectively replacing the first-rank oligonucleotide sets of organism 3, organism 2, and organism 5, in such a manner, and then it is checked whether a dimer is formed and whether dimer formation is reduced. That is, combination 2) of [1, 1, 2, 1, 1] (D-level: 2, D-link: 3), combination 3) of [1, 2, 1, 1, 1] (D-level: 2, D-link: 3), and combination 4) of [1, 1, 1, 1, 2] (D-level: 2, D-link: 4). Of combination 1) to combination 4) thus provided, combination 1) showed a reduction in dimer formation in view of the number of dimer links.

According to a specific embodiment of the present invention, step (c) is performed until dimer formation is reduced compared with the first reference combination. For example, all the combinations 1) to 4) are not provided, but while each combination is provided, it is checked whether a dimer is formed, and then when the dimer formation is reduced compared with the first reference combination, an additional combination of oligonucleotide sets is not further provided. Particularly, since the number of dimer links in the combination 1) is decreased compared with the first reference combination, only combination 1) is provided, and combinations 2) to 4) are not provided.

According to another embodiment, the method further comprises, after step (c), c-i) replacing the replaced oligonucleotide set in the combination of oligonucleotide sets provided in step (c) with another oligonucleotide set belonging to the same pool of oligonucleotide sets to provide a combination of oligonucleotide sets, which is different from the combination of oligonucleotide sets provided in step (c) only in the replaced another oligonucleotide set, and checking whether a dimer is formed between oligonucleotide sets of the combination and whether dimer formation is reduced compared with the first reference combination; or c-ii) performing step c-i), and considering the combination of oligonucleotide sets provided in step c-i) to be the combination of oligonucleotide sets provided in step (c) to repeat step c-i).

In the present embodiment, when dimer formation is not reduced although a combination of oligonucleotide sets is provided by replacing an oligonucleotide set with dimer formation in the first reference combination with another oligonucleotide set, a second reference combination cannot be provided in the step to be described below, and thus an additional replacement procedure is carried out.

According to still another embodiment of the present invention, in step c-i), the another oligonucleotide set is the next-rank oligonucleotide set.

According to still another embodiment, the replacement in step c-i) is carried out in the order from any one combination to any other combination of the combinations of oligonucleotide sets provided in step (c). Particularly, the replacement in step c-i) is carried out in the order of the combinations provided in step (c).

In FIG. 3, when dimer formation (specifically, the dimer level) is not reduced in combinations 1) to 4) compared with the first reference combination, the oligonucleotide sets replaced in combinations 1) to 4) are replaced with next-rank oligonucleotide sets in the order in which combinations 1) to 4) are provided, and it is checked whether a dimer is formed and whether dimer formation is reduced. In the combination 1), the second-rank oligonucleotide set is replaced with the third-rank oligonucleotide set in organism 1 to provide combination 5) of [3, 1, 1, 1, 1], and it is checked whether a dimer is formed (D-level: 2, D-link: 3) and whether dimer formation is reduced compared with the first reference combination. The dimer formation is not reduced in the combination 5) compared with the first reference combination, and thus, in the combination 2), the second-rank oligonucleotide set is replaced with the third-rank oligonucleotide set in organism 3 to provide combination 6) of [1, 1, 3, 1, 1], and it is checked whether a dimer is formed (D-level: 2, D-link: 2) and whether dimer formation is reduced compared with the first reference combination. In this manner, combination 7) of [1, 3, 1, 1, 1] from combination 3) and combination 8) of [1, 1, 1, 1, 3] from combination 4) are provided, respectively, and it is checked whether a dimer is formed and whether dimer formation is reduced.

The replacement in step c-i) is carried out until dimer formation is reduced, and if the criterion for a reduction in dimer formation is a reduction in dimer level, up to combination 7) is provided and combination 8) need not be provided.

If dimer formation is not reduced even in combinations 5) to 8) compared with the first reference combination, step c-i) is repeated by considering a combination of oligonucleotide sets provided in step c-i) to be a combination of oligonucleotide sets provided in step (c).

Step (d): In the Combination Checking for Whether the Dimer Formation is Reduced, Providing, as a Second Reference Combination, a Combination of Oligonucleotide Sets with a Reduction in Dimer Formation Compared with the First Reference Combination (140)

Then, in the combination checking for whether the dimer formation is reduced, the method of this invention provides, as a second reference combination, a combination of oligonucleotide sets with a reduction in dimer formation compared with the first reference combination.

Another feature of the present disclosure is that all the combinations that may be provided from the first reference combination are not objects of replacements, but a combination of oligonucleotide sets with a reduction in dimer formation compared with the first reference combination are provided as a new object of replacement (a new reference combination), thereby significantly decreasing the number of combinations of oligonucleotide sets to be checked for whether a dimer is formed.

Specifically, if the criterion for a reduction in dimer formation is a reduction in the number of dimer links, in FIG. 3, combination 1) of [2, 1, 1, 1, 1] (D-level: 2, D-link: 2), which is decreased in the number of dimer links compared with the first reference combination (D-level: 2, D-link: 3) of combinations 1) to 4), may be provided as a second reference combination. More specifically, when the replacement in step (c) is carried out until dimer formation is reduced compared with the first reference combination, combination 1) with a decreased number of dimer links is provided, and thus the combination 1) is provided as a second reference combination, and combinations 2) to 4) are not provided.

Alternatively, if the criterion for a reduction in dimer formation is a reduction in dimer level, combination 7) of combinations 5) to 8) is provided as a second reference combination.

More specifically, when the replacement in step c-i) is carried out until dimer formation is reduced, the combination 7) is provided as the second reference combination. In such a case, combination 8) need not be provided.

According to another embodiment of the present invention, the reduction in dimer formation in step (d) is a reduction to a predetermined value or less. The present embodiment shows that in step (d), not only when the dimer formation is reduced compared with the first reference combination but also the level of the reduction in dimer formation is a predetermined value or less, such a combination can be provided as a second reference combination. Specifically, the predetermined value may be selected from 1 to 8.

For example, in a case of the predetermined value being 1, the dimer formation being expressed as a dimer level, the dimer level of the first reference combination being 3, and the dimer level of the combination of oligonucleotide sets provided in step (c) being reduced to 2, the combination provided in step (c) is reduced in the dimer level compared with the first reference combination but the dimer level is not reduced to 1 or less, and thus cannot be provided as a second reference combination.

According to still more another embodiment, steps (c) and (d) are performed by the following steps:

c-1) replacing an oligonucleotide set with a dimer link in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether the dimer level is reduced compared with the first reference combination;

d-1) in the combination checking for whether the dimer level is reduced, providing, as a 1-1 reference combination, a combination of oligonucleotide sets with a reduction in dimer level compared with the first reference combination;

c-2) replacing an oligonucleotide set with a dimer link in the 1-1 reference combination with another oligonucleotide set belonging to the same pool of oligonucleotide sets to provide a combination of oligonucleotide sets, which is different from the 1-1 reference combination only in the replaced another oligonucleotide set, and checking whether a dimer is formed between oligonucleotide sets of the combination and whether the number of dimer links is decreased compared with the 1-1 reference combination; and

d-2) in the combination checking for whether the number of dimer links is decreased, providing, as the second reference combination, a combination of oligonucleotide sets with a decrease in the number of dimer links compared with the 1-1 reference combination.

The present embodiment shows that, based on a reduction in dimer formation, priority is given to the dimer level and the number of dimer links, so when the dimer level is reduced in a combination of oligonucleotide sets provided by replacement from the first reference combination, the combination is provided as a 1-1 reference combination, which is a new reference combination, and when the number of dimer links is decreased in a combination of oligonucleotide sets provided by replacement from the 1-1 reference combination, the combination is provided as a second reference combination, which is a new combination.

According to still another embodiment of the present invention, in each of steps c-1) and c-2), the another oligonucleotide set is a next-rank oligonucleotide set.

According to still another embodiment, step c-1) is performed in the order from any one oligonucleotide set to any other oligonucleotide set of the oligonucleotide sets with a dimer link in the first reference combination, and step c-2) is performed in the order from any one oligonucleotide set to any other oligonucleotide set of the oligonucleotide sets with a dimer link in the 1-1 reference combination.

According to still another embodiment of the present invention, the replacement in step c-1) is performed until the dimer level is reduced compared with the first reference combination.

According to still another embodiment, step c-2) is performed until the number of dimer links is decreased compared with the 1-1 reference combination.

According to still another embodiment of the present invention, the method further comprises, after step c-1), c-1-i) replacing the oligonucleotide set replaced in the combination of oligonucleotide sets provided in step c-1) with another oligonucleotide set belonging to the same pool of oligonucleotide sets to provide a combination of oligonucleotide sets, which is different from the combination of oligonucleotide sets provided in step c-1) only in the replaced another oligonucleotide, and checking whether a dimer is formed between oligonucleotide sets of the combination and whether the dimer level is reduced compared with the first reference combination; or c-1-ii) performing step c-1-i), and considering the combination of oligonucleotide sets provided in step c-1-i) to be the combination of oligonucleotide sets provided in step c-1) to repeat step c-1-i).

The present embodiment shows that in step c-1), an additional replacement procedure is carried out when there is no combination with a reduction in dimer level compared with the first reference combination. Specifically, the oligonucleotide set replaced in step c-1) is replaced with the next-rank oligonucleotide set, and it is checked whether the dimer level is reduced compared with the first reference combination (step c-1-i).

According to still another embodiment, in step c-1-i), the another oligonucleotide set is a next-rank oligonucleotide set.

According to still another embodiment of the present invention, the replacement in step c-1-i) is carried out in the order from any one combination to any other combination of the combinations of oligonucleotide sets provided in step c-1).

According to still another embodiment, the method further comprises, after step c-2), c-2-i) replacing the replaced oligonucleotide set in the combination of oligonucleotide sets provided in step c-2) with another oligonucleotide set belonging to the same pool of oligonucleotide sets to provide a combination of oligonucleotide sets, which is different from the combination of oligonucleotide sets provided in step c-2) only in the replaced another oligonucleotide set, and checking whether a dimer is formed between oligonucleotide sets of the combination and whether the number of dimer links is decreased compared with the 1-1 reference combination; or c-2-ii) performing step c-2-i), and considering the combination of oligonucleotide sets provided in step c-2-i) to be the combination of oligonucleotide sets provided in step c-2) to repeat step c-2-i).

The present embodiment shows that in step c-2), an additional replacement procedure is carried out when there is no combination with a decrease in the number of dimer links compared with the 1-1 reference combination. Particularly, the oligonucleotide set replaced in step c-2) is replaced with the next-rank oligonucleotide set, and it is checked whether the number of dimer links is decreased compared with the 1-1 reference combination (step c-2-i).

According to still another embodiment of the present invention, in step c-2-i), the another oligonucleotide set is a next-rank oligonucleotide set.

According to still another embodiment, the replacement in step c-2-i) is carried out in the order from any one combination to any other combination in the combinations of oligonucleotide sets provided in step c-2).

Referring to FIGS. 3 and 4, a procedure is described as follows, in which from the first reference combination, an oligonucleotide set with a dimer link is replaced to provide a combination of oligonucleotide sets with a reduction in dimer level as a 1-1 reference combination, which is a new reference combination, and from the 1-1 reference combination, an oligonucleotide set with a dimer link is replaced to provide a combination of oligonucleotide sets with a decrease in the number of dimer links as a second reference combination, which is a new reference combination.

In FIG. 3, the combination of [1, 1, 1, 1, 1] is provided as the first reference combination, and the number of dimer links (D-link) is 3 and the dimer level (D-level) is 2, and the numbers of individual dimer links of oligonucleotide sets for organisms 1 to 5 are 2, 1, 2, 0, and 1, respectively.

First, a 1-1 reference combination, which is a new reference combination with a reduction in dimer level, is provided from the first reference combination.

Specifically, when the replacement in the order from the largest in the number of individual dimer links is carried out in the first reference combination, the first-rank oligonucleotide set for organism 1 is replaced with a second-rank oligonucleotide belonging to the same pool of oligonucleotide sets to provide combination 1) of [2, 1, 1, 1, 1], and then it is checked whether a dimer is formed in combination 1) (D-level: 2, D-link: 2) and whether the dimer level is reduced compared with the first reference combination. Since the dimer level is not reduced, until a dimer level is reduced, the first-rank oligonucleotide sets of organism 3, organism 2, and organism 5 are replaced to provide combinations, respectively, and it is checked whether a dimer is formed and whether dimer formation is reduced: That is, combination 2) of [1, 1, 2, 1, 1] (D-level: 2, D-link: 3), combination 3) of [1, 2, 1, 1, 1] (D-level: 2, D-link: 3), and combination 4) of [1, 1, 1, 1, 2] (D-level: 2, D-link: 4). Since there is no combination with a reduction in dimer level of combinations 1) to 4) thus provided, an additional replacement procedure is carried out.

In the order from the combination 1) to combination 4), the second-rank oligonucleotide set is replaced with a third-rank oligonucleotide set of organism 1 in the combination 1) to provide combination 5) of [3, 1, 1, 1, 1], and it is checked whether a dimer is formed (D-level: 2, D-link: 3) and whether the dimer level is reduced compared with the first reference combination. Since the dimer level is not reduced compared with the first reference combination, combination 6) of [1, 1, 3, 1, 1] is provided from the combination 2), and it is checked whether a dimer is formed (D-level: 2, D-link: 2) and whether the dimer level is reduced compared with the first reference combination. Since the dimer level is not reduced, combination 7) of [1, 3, 1, 1, 1] is provided from the combination 3), and it is checked whether a dimer is formed (D-level: 1, D-link: 2) and whether the dimer level is reduced compared with the first reference combination. As a result, the combination 7) of [1, 3, 1, 1, 1] is reduced compared with the first reference combination in view of the dimer level, and thus the combination 7) is provided as a new reference combination, a 1-1 reference combination. When the new reference combination is provided, combination 8) of [1, 1, 1, 1, 3] is not provided and it is not checked whether a dimer is formed.

Then, a second reference combination, which is a new reference combination with a decrease in the number of dimer links, is provided from the 1-1 reference combination.

Referring to FIG. 4, in the combination 7) of [1, 3, 1, 1, 1] (D-level: 1, D-link: 2, the number of individual dimer links: 2, 0, 1, 0, 1), which is the 1-1 reference combination, oligonucleotide sets with a dimer link are replaced in the order of the largest in the number of individual dimer links, that is, in the order of oligonucleotide sets for organisms 1, 3, and 5, and it is checked whether a dimer is formed and whether the dimer level is reduced compared with the 1-1 reference combination. Combination 9) of [2, 3, 1, 1, 1] is provided from the combination 7) of [1, 3, 1, 1, 1], and it is checked whether a dimer is formed (D-level: 1, D-link: 2) and whether the number of dimer links is decreased. As a result, the combination 9) is not improved compared with combination 7) in view of the number of dimer links, and thus combination 10) of [1, 3, 2, 1, 1] is provided from the combination 7), and it is checked whether a dimer is formed (D-level: 1, D-link: 1) and whether the number of dimer links is decreased. As a result, the combination 10) improved in view of the number of dimer links is provided as a new reference combination, the second reference combination.

According to another embodiment of the present invention, steps (c) and (d) are performed by the following steps:

c) replacing an oligonucleotide set with a dimer link in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether the number of dimer links is decreased compared with the first reference combination; and

(d) in the combination checking for whether the number of dimer links is decreased, providing, as the second reference combination, a combination of oligonucleotide sets with a decrease in the number of dimer links compared with the first reference combination.

The present embodiment shows that as a result of checking whether a dimer is formed in the first reference combination, when the dimer level of the first reference combination is a predetermined value or less, or the dimer level of the first reference combination is 1, a new reference combination is provided based on the reduction in the number of dimer links.

Specifically, suppose that the predetermined value or less of the dimer level of the first reference combination is set to 2 or less. Since the dimer level of the first reference combination is 2 in FIG. 3, a new reference combination can be provided based on not a reduction in dimer level but a decrease in a dimer link among the combinations of oligonucleotide sets provided by replacement from the first reference combination. For example, the combination 1) shows a decrease in the number of dimer links compared with the first reference combination, and thus the combination 1) can be provided as a second reference combination, which is a new reference combination. In such a case, combinations 2) to 8) need not be provided.

The predetermined value of the dimer level is 7, 6, 5, 4, 3, 2, or 1, and more specifically, 2 or 1.

According to still another embodiment, in step (c), the another oligonucleotide set is a next-rank oligonucleotide set.

According to still another embodiment of the present invention, step (c) is performed in the order from any one oligonucleotide set to any other oligonucleotide set of the oligonucleotide sets with dimer link in the first reference combination.

According to still another embodiment, step (c) is performed until the number of dimer links is decreased compared with the first reference combination.

According to another embodiment of the present invention, the method further comprises, after step (d), d-i) considering the second reference combination in step (d) to be the first reference combination in step (c) to repeat steps (c) and (d) until dimer formation is reduced compared with the considered first reference combination.

The present embodiment may be applied when the second reference combination is provided by carrying out the replacement in the first reference combination to reduce dimer formation, and the dimer formation is further reduced from the second reference combination. For example, when the dimer level of the first reference combination is 3 and as a result of performing above-described steps (c) and (d), a combination with a dimer level of 2 is provided as the second reference combination, which is a new reference combination, above-described steps (c) and (d) may be repeated until the dimer level is reduced to smaller than 2 while considering the combination with a dimer level of 2 to be the first reference combination again.

More specifically, when steps (c) and (d) are the above-described steps c-1), d-1), c-2), and d-2), the method further comprises, after step d-1), d-i) considering the 1-1 reference combination in step d-1) to be the first reference combination in step c-1) to repeat steps c-1) and d-1) until the dimer level is reduced compared with the considered first reference combination; and/or, after step d-2), d-ii) considering the second reference combination in step d-2) to be the 1-1 reference combination in step c-2) to repeat steps c-2) and d-2) until the number of dimer links is decreased compared with the considered 1-1 reference combination.

Step (e): Replacing an Oligonucleotide Set with Dimer Formation in the Second Reference Combination to Provide a Combination of Oligonucleotide Sets, and Checking Whether a Dimer is Formed (150)

Then, the method of the present invention replaces an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and checks whether a dimer is formed between oligonucleotide sets of the combination.

Step (e) of the present invention is different from step (c) of the present invention in view of a reference combination, which is an object of replacement. Particularly, the object of replacement in step (c) of the present invention is the first reference combination, but the object of replacement in step (e) of the present invention is the second reference combination, which is a new reference combination provided by having a reduction in dimer formation from the first reference combination.

In step (c) of the present invention, with respect to step (d), it is checked whether a dimer is formed and whether dimer formation is improved compared with the first reference combination. With respect to the above-described steps (c) and (d), as a result of checking dimer formation in the combination of oligonucleotide sets provided in step (c), a combination with all dimers removed can be provided as a combination for detecting a plurality of target nucleic acid molecules by step (f), without the need to perform steps (d) and (e). Therefore, the combination provided as the second reference combination in step (d) represents a combination in which a dimer is formed but the dimer formation is reduced compared with the first reference combination, as a result of checking whether a dimer is formed in step (c).

On the contrary, with respect to step (f) of the present invention to be described below, the checking of whether a dimer is formed in step (e) is carried out by confirming whether all the dimers are not formed in the combination provided by replacement from the second reference combination. The provision of a new reference combination through the criterion for a reduction in dimer formation is performed in steps (c) and (d), and a combination used to detect a plurality of target nucleic acid molecules is provided in steps (e) and (f). If all the dimers are not removed even in step (e), a combination in which all the dimers are removed is provided through an additional replacement procedure from the second reference combination.

Excluding such differences, the common descriptions between the above-described step (c) and step (e) are omitted in order to avoid undue redundancy leading to the complexity of this specification.

According to another embodiment, in step (e), the another oligonucleotide set is a next-rank oligonucleotide set.

According to another embodiment of the present invention, in step (e), the oligonucleotide set with dimer formation is an oligonucleotide set with a dimer link.

According to another embodiment, the replacement in step (e) is carried out in the order from any one oligonucleotide set to any other oligonucleotide set of the oligonucleotide sets with dimer formation in the second reference combination.

According to still another embodiment of the present invention, the order from any one oligonucleotide set to any other oligonucleotide set in step (e) is (i) an order from an oligonucleotide set with the largest number of individual dimer links to an oligonucleotide set with the smallest number of individual dimer links, and (ii) when the number of individual dimer links is the same, an order from an oligonucleotide set located in the front of a combination to an oligonucleotide set located in the back or an order from an oligonucleotide set belonging to a pool of oligonucleotide sets including the largest number of oligonucleotide sets to an oligonucleotide set belonging to a pool of oligonucleotide sets including the smallest number of oligonucleotide sets.

According to another embodiment of the present invention, step (e) is performed until a dimer is not formed from the second reference combination.

In FIG. 4, combination 10) of [1, 3, 2, 1, 1] is the second reference combination provided in step (d). The combination has a dimer link between the oligonucleotide sets for organism 1 and organism 5, and in the combination, the dimer level (D-level) is 1 and the number of dimer links (D-links) is 1. In the second reference combination, which is a new reference combination, the oligonucleotide set having a dimer link in organism 1 is replaced with a next-rank oligonucleotide set to provide combination 11) of [2, 3, 2, 1, 1], and as a result of checking whether a dimer is formed, all the dimers are not removed (D-level: 1, D-link: 1). Therefore, the oligonucleotide set with a dimer link in organism 5 is replaced with a next-rank oligonucleotide set to provide combination 12) of [1, 3, 2, 1, 2], and as a result of checking whether a dimer is formed, no dimer formation is confirmed. Therefore, combination 12) can be provided as a combination of oligonucleotide sets used to simultaneously detect a plurality of target nucleic acid molecules.

According to another embodiment of the present invention, the method further comprises, after step (e), e-i) replacing the replaced oligonucleotide set in the combination of oligonucleotide sets provided in step (e) with another oligonucleotide set belonging to the same pool of oligonucleotide sets to provide a combination of oligonucleotide sets, which is different from the combination of oligonucleotide sets provided in step (e) only in the replaced another oligonucleotide, and checking whether a dimer is formed between oligonucleotide sets of the combination; or e-ii) performing step e-i), and considering the combination of oligonucleotide sets provided in step e-i) to be the combination of oligonucleotide sets provided in step (e) to repeat step e-i).

The present embodiment shows that when a combination with no dimer formation is not provided even through the replacement of an oligonucleotide set with dimer formation with another oligonucleotide set in the second reference combination, an additional replacement procedure is carried out.

According to still another embodiment, in step e-i), the another oligonucleotide set is a next-rank oligonucleotide set.

According to still another embodiment of the present invention, step e-i) is performed in the order from any one combination to any other combination in the combinations of oligonucleotide sets provided in step (e).

Step (f): In the Combination Checking for Whether a Dimer is Formed, Providing a Combination of Oligonucleotide Sets with No Dimer Formation (160)

Lastly, the method of the present invention provides a combination of oligonucleotide sets with no dimer formation in the combination confirming whether a dimer is formed. The combination of oligonucleotide sets with no dimer formation is used to simultaneously detect a plurality of target nucleic acid molecules.

In FIG. 4, the combinations are provided from combination 10), which is the second reference combination, by replacing the oligonucleotide sets with a dimer link in organisms 1 and 5 with next-rank oligonucleotide sets, respectively, and as a result of checking whether a dimer is formed, there is no dimer formation in combination 12) [1, 3, 2, 1, 2], and thus combination 12) is provided as a combination of oligonucleotide sets used to simultaneously detect a plurality of target nucleic acid molecules.

In this manner, an optimal combination of oligonucleotide sets used to simultaneously detect a plurality of target nucleic acid molecules can be provided.

### Storage Medium, Device, and Program

In another aspect of the present invention, there is provided a computer readable storage medium containing indications to configure a processor to perform a method for preparing an optimal combination of oligonucleotide sets used to simultaneously detect a plurality of target nucleic acid molecules, the method comprising: (a) providing a pool of oligonucleotide sets used to detect each of the plurality of target nucleic acid molecules for each of the plurality of target nucleic acid molecules; wherein the oligonucleotide sets each comprises one or more oligonucleotides, (b) providing, as a first reference combination, a combination of oligonucleotide sets combined from the pool of oligonucleotide sets provided for each of the plurality of target nucleic acid molecules, and checking whether a dimer is formed between oligonucleotide sets of the combination; (c) replacing an oligonucleotide set with dimer formation in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether dimer formation is reduced compared with the first reference combination; (d) in the combination checking for whether the dimer formation is reduced, providing, as a second reference combination, a combination of oligonucleotide sets with a reduction in dimer formation compared with the first reference combination; (e) replacing an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and checking whether a dimer is formed between oligonucleotide sets of the combination; and (f) in the combination checking for whether the dimer is formed, providing a combination of oligonucleotide sets with no dimer formation; wherein the combination of oligonucleotide sets with no dimer formation is used to simultaneously detect the plurality of target nucleic acid molecules.

In still another aspect of the present invention, there is provided a computer program to be stored on a computer readable storage medium, to configure a processor to perform a method for preparing an optimal combination of oligonucleotide sets used to simultaneously detect a plurality of target nucleic acid molecules, the method comprising: (a) providing a pool of oligonucleotide sets used to detect each of the plurality of target nucleic acid molecules for each of the plurality of target nucleic acid molecules; wherein the oligonucleotide sets each comprises one or more oligonucleotides, (b) providing, as a first reference combination, a combination of oligonucleotide sets combined from the pool of oligonucleotide sets provided for each of the plurality of target nucleic acid molecules, and checking whether a dimer is formed between oligonucleotide sets of the combination; (c) replacing an oligonucleotide set with dimer formation in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether dimer formation is reduced compared with the first reference combination; (d) in the combination checking for whether the dimer formation is reduced, providing, as a second reference combination, a combination of oligonucleotide sets with a reduction in dimer formation compared with the first reference combination; (e) replacing an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and checking whether a dimer is formed between oligonucleotide sets of the combination; and (f) in the combination checking for whether the dimer is formed, providing a combination of oligonucleotide sets with no dimer formation; wherein the combination of oligonucleotide sets with no dimer formation is used to simultaneously detect the plurality of target nucleic acid molecules.

In another aspect of the present invention, there is provided a device for preparing an optimal combination of oligonucleotide sets used to simultaneously detect a plurality of target nucleic acid molecules, comprising (a) a computer processor and (b) the computer readable storage medium of the present invention coupled to the computer processor.

Since the storage medium, the device, and the computer program of the present invention are intended to perform the present methods described hereinabove in a computer, the common descriptions between them are omitted in order to avoid undue redundancy leading to the complexity of this specification.

The program instructions are operative, when preformed by the processor, to cause the processor to perform the method of the present invention described above. The program instructions for performing a method for preparing an optimal combination of oligonucleotide sets may comprise the following instructions: (i) an instruction to provide a pool of oligonucleotide sets used to detect each of the plurality of target nucleic acid molecules for each of the plurality of target nucleic acid molecules; (ii) an instruction to provide, as a first reference combination, a combination of oligonucleotide sets combined from the pool of oligonucleotide sets provided for each of the plurality of target nucleic acid molecules, and check whether a dimer is formed between oligonucleotide sets of the combination; (iii) an instruction to replace an oligonucleotide set with dimer formation in the first reference combination with another oligonucleotide set belonging to the same pool of oligonucleotide sets to provide a combination of oligonucleotide sets, which is different from the first reference combination only in the replaced another oligonucleotide set, and check whether a dimer is formed between oligonucleotide sets of the combination and whether dimer formation is reduced compared with the first reference combination; (iv) an instruction to, in the combination checking for whether the dimer formation is reduced, provide, as a second reference combination, a combination of oligonucleotide sets with a reduction in dimer formation compared with the first reference combination; (v) an instruction to replace an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and check whether a dimer is formed between oligonucleotide sets of the combination; and (vi) an instruction to, in the combination checking for whether the dimer is formed, provide (e.g., display on an output device) a combination of oligonucleotide sets with no dimer formation.

The method of the present invention is implemented in a processor, and the processor may be a processor in a stand-alone computer, a network attached computer, or a data acquisition device such as a real-time PCR machine.

The types of the computer readable storage medium include various storage mediums known in the art, such as CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory, and web server, but are not limited thereto.

An optimal combination of oligonucleotide sets may be provided in various manners. For example, the optimal combination of oligonucleotide sets may be provided to a separate system, such as a desktop computer system, via a network connection (e.g., LAN, VPN, intranet, and internet) or a direct connection (e.g., USB or other direct wired or wireless connection), or may be provided on a portable medium, such as CD, DVD, floppy disk, or portable HDD. Similarly, the optimal combination of oligonucleotide sets may be provided to a server system via a network connection (e.g., LAN, VPN, Internet, intranet, and wireless communication network) to a client, such as a notebook or a desktop computer system.

The instructions to configure the processor to perform the present invention may be included in a logic system. The instructions may be downloaded and stored in a memory module (e.g., hard drive or other memory such as a local or attached RAM or ROM), although the instructions can be provided on any software storage medium, such as portable HDD, USB, floppy disk, CD and DVD. A computer code for implementing the present invention may be implemented in a variety of coding languages, such as C, C++, Java, Visual Basic, VBScript, JavaScript, Perl, and XML. In addition, a variety of languages and protocols may be used in external and internal storage and transmission of data and commands according to the present invention.

The computer processor may be constructed in such a manner that a single processor can make several performances. Alternatively, the processor unit may be constructed in such a manner that several processors make several performances, respectively.

The features and advantages of this invention are summarized as follows.

(a) In preparing (or providing) an optimal combination of oligonucleotide sets used to detect a plurality of target nucleic acid molecules, the conventional method checked whether a dimer is formed in all the candidate combinations of oligonucleotide sets combined from each pool of oligonucleotide sets. Such a conventional method is possible when the size of a pool of oligonucleotide sets is small and the number of target nucleic acid molecules to be detected is small, but when the size of a pool of oligonucleotide sets is large and the number of target nucleic acid molecules to be detected is large, the conventional method consumes a long time in checking whether a dimer is formed, to thereby provide an optimal combination of oligonucleotide sets or even cannot provide an optimal combination.

(b) In the present invention, to solve the problems of the above-described conventional method, only an oligonucleotide set with dimer formation is replaced in the first reference combination of oligonucleotide sets to provide, as a new reference combination, a combination with a reduction in dimer formation compared with the first reference combination, and then only an oligonucleotide set with dimer formation is replaced in the new reference combination to provide a combination with all dimers removed, so that the number of oligonucleotide sets and candidate combinations to be checked for whether a dimer is formed were significantly reduced.

(c) Therefore, the present invention can provide a combination of oligonucleotide sets with no interference between oligonucleotide sets, which are used to detect a plurality of target nucleic acid molecules, with speed and accuracy.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Providing Optimal Combinations of Oligonucleotide Sets for Diagnosis of Dengue Virus Antibiotic Resistance

It is intended to provide up to 10 optimal combinations of oligonucleotide sets used to simultaneously detect four organisms (analytes) for diagnosis of Dengue virus antibiotic resistance by running once an algorithm of providing an optimal combination with no dimer between oligonucleotide sets (OSs).

### (1) Providing Each Pool of Oligonucleotide Sets

Primers and probes were designed, and primer pairs and probes were combined to provide a pool of oligonucleotide sets for each of the four organisms so that they could be used to detect each of the four organisms. 614 oligonucleotide sets were provided for organism 1 (analyte 1; A1), 952 for organism 2 (analyte 2; A2), 1493 for organism 3 (analyte 3; A3), 1012 for organism 4 (analyte 4; A4), and 100000 for an internal control (IC). The oligonucleotide sets included in the pool of oligonucleotide sets were given ranks through ranking for each of the following priority items: (i) the total sum of the number of oligonucleotides contained in an oligonucleotide set; the smaller the total sum, the higher the priority; (ii) the total sum of the number of a degenerate base and/or universal base introduced into an oligonucleotide contained in an oligonucleotide set; the smaller the total sum, the higher the priority; (iii) a target-coverage of an oligonucleotide set for a plurality of target nucleic acid sequences of a target nucleic acid molecule; the larger the target-coverage, the higher the priority; and (iv) the total sum of the number of oligonucleotide patterns generated by a degenerate base introduced into an oligonucleotide contained in an oligonucleotide set; the smaller the total sum, the higher the priority. The four priority items have a difference in criticality, and priority item (i) has the highest criticality, and subsequently, the criticality was assigned up to priority item (v) in order. Each of the oligonucleotide sets comprises at least one primer pair and at least one probe.

### (2) Providing Optimal Combination 1 of Oligonucleotide Sets

A combination of oligonucleotide sets having the minimum rank sum was provided as a first reference combination from the pool of oligonucleotide sets for each of four organisms. In addition, it was checked whether a dimer is formed in the first reference combination. As a result, no dimer formation was confirmed in the first reference combination, and the first reference combination was selected as an optimal combination 1 of oligonucleotide sets. Then, an oligonucleotide set for IC was added to the optimal combination 1 in the order of rank, and it was checked whether a dimer was formed. As a result, no dimer formation was confirmed in the combination in which a first-rank oligonucleotide set for IC was added to the optimal combination 1, and such a combination was summarized in Table 1 below. The checking of whether a dimer is formed was carried out by determining whether at least one criterion of the following criteria is met: (i) the proportion of total nucleotides forming Watson-Crick base pairs between oligonucleotides is 40% or more; and (ii) the proportion of consecutive nucleotides forming Watson-Crick base pairs between oligonucleotides is 20% or more.

TABLE 1

| Analyte | Rank | Type | Strand | Start | End | Sequence |
|---|---|---|---|---|---|---|
| A1 | 1 | Primer | Forward (F) | 837 | 877 | AGGATAGAGAAYATAAAAMATGAACAIIIIICAACATGGC |
| | | | | | | (SEQ ID NO: 1) |
| | 1 | Primer | F | 735 | 768 | CTCACAGGAARCCAACRTATIIIIIAGACGTGGA |
| | | | | | | (SEQ ID NO: 2) |
| | 1 | Probe | Reverse (R) | 944 | 970 | ACCATRGATGAGGCTGATCCTGATGGC |
| | | | | | | (SEQ ID NO: 3) |
| | 1 | Probe | R | 911 | 940 | ACCTCATATGATCCRTGRTAGGCCCATGT |
| | | | | | | (SEQ ID NO: 4) |
| | 1 | Primer | R | 1036 | 1068 | CCTCTGTTGTCCAAAGGGTIIIIIGTCAGTCAT |
| | | | | | | (SEQ ID NO: 5) |
| | 1 | Primer | R | 1005 | 1038 | CATRGCTATTTGTGTGACCAIIIIIATRACATCC |
| | | | | | | (SEQ ID NO: 6) |
| A2 | 1 | Primer | F | 63 | 110 | ACCGCGTGTCRACTGTRIIIIIGCTGACAAAG |
| | | | | | | (SEQ ID NO: 7) |
| | 1 | Primer | F | 39 | 76 | CAATATGCTGAAACGCGAGAGIIIIIGCGTGTCRA |
| | | | | | | (SEQ ID NO: 8) |
| | 1 | Probe | F | 122 | 144 | TGGAATGCTGCARGGACGMGG |
| | | | | | | (SEQ ID NO: 9) |
| | 1 | Probe | R | 155 | 176 | TGCCACMARGGCCATGAACAG |
| | | | | | | (SEQ ID NO: 10) |
| | 1 | Probe | R | 184 | 210 | TGCTGTTGGYGGGATTGTYAGGAAAC |
| | | | | | | (SEQ ID NO: 11) |
| | 1 | Primer | R | 303 | 333 | GCDGTYCTGCGTCTCCTIIIIIAGATGTTCA |
| | | | | | | (SEQ ID NO: 12) |
| | 1 | Primer | R | 334 | 369 | ATCRCTGTTGGAATCAGCAIIIIIATCAYGCCT |
| | | | | | | (SEQ ID NO: 13) |
| | 1 | Primer | R | 271 | 306 | TTCARCATCCTTCCRATCTCTIIIIIGAACCCTCTC |
| | | | | | | (SEQ ID NO: 14) |
| A3 | 1 | Primer | F | 1358 | 1393 | TGATGGGYAAGAGAGAGAARAIIIIIGGAGAGTTTG |
| | | | | | | (SEQ ID NO: 15) |
| | 1 | Probe | F | 1421 | 1444 | GGTACATGTGGTTRGGAGCYAGGT |
| | | | | | | (SEQ ID NO: 16) |
| | 1 | Primer | R | 1595 | 1631 | TCYTCTGTTATTCTTGTRTCCCIIIIIGCTGTGTCAT |
| | | | | | | (SEQ ID NO: 17) |
| A4 | 1 | Primer | F | 33 | 66 | TTTCAATATGCTGAAACGCGIIIIIAACCGCGTA |
| | | | | | | (SEQ ID NO: 18) |
| | 1 | Primer | F | 35 | 67 | TCAATATGCTGAAACGCGMIIIIIACCGCGTMT |
| | | | | | | (SEQ ID NO: 19) |
| | 1 | Probe | F | 80 | 112 | GGTTGGTGAAGAGATTCTCRACHGGAC |
| | | | | | | (SEQ ID NO: 20) |
| | 1 | Probe | F | 121 | 146 | GGGAAAGGACCCTTACGGATGGTGYT |
| | | | | | | (SEQ ID NO: 21) |
| | 1 | Primer | R | 169 | 199 | GAATCCCTGCTGTTGGYIIIIIGGAAAGRAC |
| | | | | | | (SEQ ID NO: 22) |
| | 1 | Primer | R | 246 | 278 | ARCATGCGRCCTATCTCCTIIIIIAATCCAATC |
| | | | | | | (SEQ ID NO: 23) |
| IC | 1 | Primer | F | 302 | 335 | GCCTTTCTCATTCGTTGTCIIIIIGCTCCTACCT |
| | | | | | | (SEQ ID NO: 24) |
| | 1 | Probe | F | 431 | 452 | CGTTGACGCTCCCTACGGGTGG |
| | | | | | | (SEQ ID NO: 25) |
| | 1 | Primer | R | 477 | 507 | CCCCTCGATGCATGGCIIIIITTGGGCCTT |
| | | | | | | (SEQ ID NO: 26) |

(3) Providing Each Pool of Oligonucleotide Sets with Duplicate Oligonucleotide Sets Removed

To ensure diversity of the optimal combination of oligonucleotide sets, oligonucleotide sets containing oligonucleotides, which are the same as some or all of oligonucleotides contained in the oligonucleotide sets of the combination provided in (2) above, were removed from each pool of oligonucleotide sets in (1) above. If, as a result of removing oligonucleotide sets containing the same oligonucleotides, no oligonucleotide set was present in a pool of oligonucleotide sets, the oligonucleotide sets included in the combination provided in (2) above were again used.

As a result of removing oligonucleotide sets containing oligonucleotides, which are more than 50% the same as the oligonucleotides contained in the oligonucleotide sets of the combination provided in (2) above, from each pool of oligonucleotide sets in (1) above, oligonucleotide sets for organism 1 were reduced to 527, oligonucleotide sets for organism 2 to 382, oligonucleotide sets for organism 3 to 1,349, oligonucleotide sets for organism 4 to 526, and oligonucleotide sets for IC to 92689.

### (4) Providing Optimal Combination 2 of Oligonucleotide Sets

A combination of oligonucleotide sets having the minimum rank sum from a pool of oligonucleotide sets for each of organisms 1 to 4 provided in (3) above, was provided as a first reference combination, and it was checked whether a dimer is formed. A combination in which an oligonucleotide set with a dimer link in the first reference combination was replaced with a next-rank oligonucleotide set was provided, and it was checked whether a dimer is formed and whether the dimer level is reduced compared with the first reference combination. When there was no combination with a reduction in dimer level, a combination in which only the replaced oligonucleotide set was replaced was provided and it was checked whether a dimer is formed and whether the dimer level is reduced compared with the first reference combination. When the dimer level was reduced compared with the first reference combination, an additional combination was not provided, and the combination with a reduction in dimer level was provided as a new reference combination (a 1-1 reference combination). When the dimer level of the new reference combination had a predetermined level (D-level: 2) or less, a decrease in the number of dimer links was set as a criterion for providing a new reference combination, and an oligonucleotide set with a dimer link was replaced. The replacement in the procedure of providing a new reference combination (a 1-1 reference combination) from the first reference combination on the basis of a reduction in dimer level is the same as the replacement in the procedure of providing a new reference combination (a second reference combination) on the basis of a decrease in the number of dimer links. When the decrease in the number of dimer links is a predetermined value (D-link: 1) or less, an oligonucleotide set with a dimer link was replaced with a next-rank oligonucleotide set in the same manner of the above-described replacement procedure, thereby selecting, as optimal combination 2, a combination of oligonucleotide sets with all dimers removed, that is, no dimer formation. In addition, each oligonucleotide set for IC was added to the optimal combination in the order of rank, and it was checked whether a dimer is formed, and the combination with no dimer formation was summarized in Table 2 below.

TABLE 2

| Analyte | Rank | Type | Strand | Start | End | Sequence |
|---|---|---|---|---|---|---|
| A1 | 6 | Primer | Forward (F) | 1316 | 1347 | GCAHARAGAGAGGGAGCTIIIIIAACAGGGAA |
| | | | | | | (SEQ ID NO: 27) |
| | 6 | Primer | F | 1261 | 1296 | AYCAATGGAACTCAGCAAAAGIIIIIGTGGAAGAYG |
| | | | | | | (SEQ ID NO: 28) |
| | 6 | Probe | Reverse (R) | 1426 | 1452 | TCCCARCCACATGTACCATATTGCRCG |
| | | | | | | (SEQ ID NO: 29) |
| | 6 | Probe | R | 1354 | 1380 | TCCCCATCATGTTGTARACACACGTRG |
| | | | | | | (SEQ ID NO: 30) |
| | 6 | Primer | R | 1488 | 1521 | TGAATTCTCTCTGCTGAACCIIIIITCTTCGTTC |
| | | | | | | (SEQ ID NO: 31) |
| | 6 | Primer | R | 1479 | 1513 | CTCTACTGAACCAGTGATCTIIIIICATGAAACCA |
| | | | | | | (SEQ ID NO: 32) |
| | 6 | Primer | R | 1514 | 1546 | GTCCTTCTCCYTCCACTCCIIIIIGTGAATTCT |
| | | | | | | (SEQ ID NO: 33) |
| A2 | 36 | Primer | F | 151 | 182 | ACCCTCATGGCCATAGAYIIIIITGARTTGTG |
| | | | | | | (SEQ ID NO: 34) |
| | 36 | Primer | F | 160 | 192 | GCCATDGACCTTGGTGAAIIIIITGAAGAYACA |
| | | | | | | (SEQ ID NO: 35) |
| | 36 | Primer | F | 214 | 247 | CTCAGGCAGAATGARCCAIIIIICATAGAYTGT |
| | | | | | | (SEQ ID NO: 36) |
| | 36 | Primer | F | 170 | 202 | TTGGTGARTTGTGTGAAGAIIIIITCACGTAYA |
| | | | | | | (SEQ ID NO: 37) |
| | 36 | Probe | F | 357 | 380 | TGGAGACACGAACTGAAACATGGA |
| | | | | | | (SEQ ID NO: 38) |
| | 36 | Probe | F | 357 | 383 | TGGAAACGAGAACTGAAACATGGATGT |
| | | | | | | (SEQ ID NO: 39) |
| | 36 | Probe | F | 377 | 400 | TGGATGTCRTCAGAAGGGGCYTGG |
| | | | | | | (SEQ ID NO: 40) |
| | 36 | Primer | R | 439 | 471 | GCCAGGATTGCTGCCATTAIIIIIAAGCCTGGA |
| | | | | | | (SEQ ID NO: 41) |
| | 36 | Primer | R | 410 | 443 | CTGGATGTCTCARGAYCCAIIIIICAATTCTCTG |
| | | | | | | (SEQ ID NO: 42) |
| | 36 | Primer | R | 449 | 483 | CCTATGGTGTATGCCARGATIIIIICCATTATGGT |
| | | | | | | (SEQ ID NO: 43) |
| A3 | 49 | Primer | F | 599 | 631 | AYGGAGGAATGCTTGTGAIIIIICCACTYTCAC |
| | | | | | | (SEQ ID NO: 44) |
| | 49 | Probe | R | 796 | 821 | TCCATRTTGGGTGTTTCTGGTTCYGC |
| | | | | | | (SEQ ID NO: 45) |
| | 49 | Primer | R | 935 | 968 | ACTCCRTTTATCATGGAGGAIIIIIAGCCTGTRG |
| | | | | | | (SEQ ID NO: 46) |
| A4 | 46 | Primer | F | 130 | 160 | CCCTTACGGATGGTGYTIIIIITCATYACGT |
| | | | | | | (SEQ ID NO: 47) |
| | 46 | Primer | F | 91 | 129 | AGATTCTCRACYGGACTTTIIIIIGGGAAAGGA |
| | | | | | | (SEQ ID NO: 48) |
| | 46 | Probe | F | 179 | 200 | TCCCACCAACAGCAGGGATTCT |
| | | | | | | (SEQ ID NO: 49) |
| | 46 | Probe | R | 250 | 274 | TGCGRCCTATCTCCTTCCTRAATCC |
| | | | | | | (SEQ ID NO: 50) |
| | 46 | Primer | R | 313 | 344 | GCCATTACGGTGGGAAYCIIIIICARCAATGT |
| | | | | | | (SEQ ID NO: 51) |
| | 46 | Primer | R | 299 | 331 | GAATCAAGCACARCARTGTIIIIITTGACCTTT |
| | | | | | | (SEQ ID NO: 52) |
| | 46 | Primer | R | 322 | 355 | ACARGTGAAACGCCATTACIIIIIGAATCAARCA |
| | | | | | | (SEQ ID NO: 53) |
| | 46 | Primer | R | 396 | 433 | GTTGTCTTRAACAAGAGAGGIIIIICCCTTTCRT |
| | | | | | | (SEQ ID NO: 54) |
| IC | 7 | Primer | F | 447 | 478 | GGGTGGACTGTGGAGAGIIIIIGCACTGCTAA |
| | | | | | | (SEQ ID NO: 55) |
| | 7 | Probe | F | 514 | 535 | CCGTATGGCCAACAACTGGCGC |
| | | | | | | (SEQ ID NO: 56) |
| | 7 | Primer | R | 548 | 583 | GCTAACGCATCTAAGGTATGGIIIIICGAGAAAGGA |
| | | | | | | (SEQ ID NO: 57) |

### (5) Providing Optimal Combinations 3 to 10 of Oligonucleotide Sets

The procedures (3) and (4) were repeated to provide optimal combinations 3 to 10 of oligonucleotide sets.

It took a total of 9.0 seconds to provide optimal combinations 1 to 10 of oligonucleotide sets by the method of Example 1.

### Example 2: Providing Optimal Combinations of Oligonucleotide Sets for Diagnosis of Respiratory Infection-Related Virus

By the same method as described in Example 1, it is intended to provide up to 10 optimal combinations of oligonucleotide sets used to simultaneously detect 11 viruses related with respiratory infection comprising adenovirus and parainfluenza virus, and an internal control (IC).

For 11 viruses and the internal control (IC), the pools of oligonucleotide sets including 591, 419, 1921, 510, 282, 1, 315, 20661, 4831, 3872, 357, and 195 oligonucleotide sets were provided, respectively.

Optimal combinations 1 to 10 of oligonucleotide sets selected and provided by the same method as in Example 1 were summarized in Table 3 below, and it took about 13 minutes to provide the 10 optimal combinations.

TABLE 3

| No. | Optimal combination of oligonucleotide sets |
|---|---|
| 1 | [66, 11, 1, 12, 100, 1, 283, 1, 1, 1, 2, 8] |
| 2 | [3, 325, 114, 7, 260, 1, 21, 11, 7, 5, 3, 64] |
| 3 | [12, 198, 29, 37, 3, 1, 177, 264, 28, 400, 14, 29] |
| 4 | [86, 86, 167, 25, 30, 1, 1, 130, 74, 522, 26, 23] |
| 5 | [14, 158, 74, 65, 16, 1, 247, 141, 589, 921, 20, 40] |
| 6 | [95, 19, 76, 27, 31, 1, 16, 295, 336, 680, 58, 45] |
| 7 | [118, 171, 173, 89, 69, 1, 126, 219, 746, 1088, 184, 12] |
| 8 | [88, 134, 311, 59, 201, 1, 78, 274, 596, 1382, 248, 51] |
| 9 | [110, 216, 73, 87, 87, 1, 18, 521, 911, 1467, 147, 55] |
| 10 | [48, 56, 234, 75, 146, 1, 115, 933, 2518, 1725, 7, 19] |

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A method for preparing an optimal combination of oligonucleotide sets used to simultaneously detect a plurality of target nucleic acid molecules, comprising:
(a) providing a pool of oligonucleotide sets used to detect each of the plurality of target nucleic acid molecules for each of the plurality of target nucleic acid molecules; wherein the oligonucleotide sets each comprises one or more oligonucleotides,
(b) providing, as a first reference combination, a combination of oligonucleotide sets combined from the pool of oligonucleotide sets provided for each of the plurality of target nucleic acid molecules, and checking whether a dimer is formed between oligonucleotide sets of the combination;
(c) replacing an oligonucleotide set with dimer formation in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether dimer formation is reduced compared with the first reference combination;
(d) in the combination checking for whether the dimer formation is reduced, providing, as a second reference combination, a combination of oligonucleotide sets with a reduction in dimer formation compared with the first reference combination;
(e) replacing an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and checking whether a dimer is formed between oligonucleotide sets of the combination; and
(f) in the combination checking for whether the dimer is formed, providing a combination of oligonucleotide sets with no dimer formation; wherein the combination of oligonucleotide sets with no dimer formation is used to simultaneously detect the plurality of target nucleic acid molecules.

2. The method according to claim 1, wherein the plurality of target nucleic acid molecules are target nucleic acid molecules of one organism or a plurality of organisms.

3. The method according to claim 1, wherein the oligonucleotide comprises a primer pair and/or a probe.

4. The method according to claim 1, wherein the oligonucleotide sets in step (a) are ranked based on predetermined sorting criteria, wherein the first reference combination in step (b) has a predetermined rank sum, and wherein in each of steps (c) and (e), the another oligonucleotide set is a next-rank oligonucleotide set.

5. The method according to claim 4, wherein the ranking of the oligonucleotide sets in step (a) is carried out by ranking based on at least one of the following predetermined sorting criteria:
(i) the total sum of the number of oligonucleotides contained in an oligonucleotide set; the smaller the total sum, the higher the priority,
(ii) the total sum of the number of a degenerate base and/or universal base introduced into an oligonucleotide contained in an oligonucleotide set; the smaller the total sum, the higher the priority,
(iii) a target-coverage of an oligonucleotide set for a plurality of target nucleic acid sequences of a target nucleic acid molecule; the larger the target-coverage, the higher the priority, and
(iv) the total sum of the number of oligonucleotide patterns generated by a degenerate base introduced into an oligonucleotide contained in an oligonucleotide set; the smaller the total sum, the higher the priority.

6. The method according to claim 4, wherein the first reference combination in step (b) has the minimum rank sum.

7. The method according to claim 1, wherein the checking of whether the dimer is formed is carried out by confirming whether one or more of the following criteria are satisfied:
(i) the proportion of total nucleotides forming Watson-Crick base pairs between oligonucleotides is a predetermined value or more; and
(ii) the proportion of consecutive nucleotides forming Watson-Crick base pairs between oligonucleotides is a predetermined value or more.

8. The method according to claim 1, wherein the dimer formation is expressed as a dimer link and/or a dimer level, the dimer link represents one or more dimer pairs formed between two oligonucleotide sets in the oligonucleotide sets of the combination and the one or more dimer pairs are considered to be one dimer link, and the dimer level represents the minimum number of oligonucleotide sets that need to be replaced in order to remove all dimer links formed between oligonucleotide sets of the combination.

9. The method according to claim 8, wherein in each of steps (c) and (e), the oligonucleotide set with dimer formation is an oligonucleotide set with a dimer link.

10. The method according to claim 1, wherein the replacement in step (c) is carried out in the order from any one oligonucleotide set to any other oligonucleotide set of the oligonucleotide sets with dimer formation in the first reference combination, and wherein the replacement in step (e) is carried out in the order from any one oligonucleotide set to any other oligonucleotide set of the oligonucleotide sets with dimer formation in the second reference combination.

11. The method according to claim 1, wherein step (c) is performed until dimer formation is reduced compared with the first reference combination.

12. The method according to claim 1, the method further comprises, after step (c), c-i) replacing the replaced oligonucleotide set in the combination of oligonucleotide sets provided in step (c) with another oligonucleotide set belonging to the same pool of oligonucleotide sets to provide a combination of oligonucleotide sets, which is different from the combination of oligonucleotide sets provided in step (c) only in the replaced another oligonucleotide set, and checking whether a dimer is formed between oligonucleotide sets of the combination and whether dimer formation is reduced compared with the first reference combination; or c-ii) performing step c-i), and considering the combination of oligonucleotide sets provided in step c-i) to be the combination of oligonucleotide sets provided in step (c) to repeat step c-i).

13. The method according to claim 8, wherein steps (c) and (d) are performed by the following steps:
c-1) replacing an oligonucleotide set with a dimer link in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether the dimer level is reduced compared with the first reference combination;
d-1) in the combination checking for whether the dimer level is reduced, providing, as a 1-1 reference combination, a combination of oligonucleotide sets with a reduction in dimer level compared with the first reference combination;
c-2) replacing an oligonucleotide set with a dimer link in the 1-1 reference combination with another oligonucleotide set belonging to the same pool of oligonucleotide sets to provide a combination of oligonucleotide sets, which is different from the 1-1 reference combination only in the replaced another oligonucleotide set, and checking whether a dimer is formed between oligonucleotide sets of the combination and whether the number of dimer links is decreased compared with the 1-1 reference combination; and
d-2) in the combination checking for whether the number of dimer links is decreased, providing, as the second reference combination, a combination of oligonucleotide sets with a decrease in the number of dimer links compared with the 1-1 reference combination.

14. The method according to claim 13, wherein the replacement in step c-1) is performed until the dimer level is reduced compared with the first reference combination.

15. The method according to claim 13, wherein the replacement in step c-2) is performed until the number of dimer links is decreased compared with the 1-1 reference combination.

16. The method according to claim 8, wherein steps (c) and (d) are performed by the following steps:
c) replacing an oligonucleotide set with a dimer link in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether the number of dimer links is decreased compared with the first reference combination; and
(d) in the combination checking for whether the number of dimer links is decreased, providing, as the second reference combination, a combination of oligonucleotide sets with a decrease in the number of dimer links compared with the first reference combination.

17. The method according to claim 16, wherein step (c) is performed until the number of dimer links is decreased compared with the first reference combination.

18. The method according to claim 1, the method further comprises, after step (d), d-i) considering the second reference combination in step (d) to be the first reference combination in step (c) to repeat steps (c) and (d) until the dimer formation is reduced compared with the considered first reference combination.

19. The method according to claim 1, wherein step (e) is performed until a dimer is not formed from the second reference combination.

20. The method according to claim 1, the method further comprises, after step (e), e-i) replacing the replaced oligonucleotide set in the combination of oligonucleotide sets provided in step (e) with another oligonucleotide set belonging to the same pool of oligonucleotide sets to provide a combination of oligonucleotide sets, which is different from the combination of oligonucleotide sets provided in step (e) only in the replaced another oligonucleotide set, and checking whether a dimer is formed between oligonucleotide sets of the combination; or e-ii) performing step e-i), and considering the combination of oligonucleotide sets provided in step e-i) to be the combination of oligonucleotide sets provided in step (e) to repeat step e-i).

21. A computer readable storage medium containing indications to configure a processor to perform a method for preparing an optimal combination of oligonucleotide sets used to simultaneously detect a plurality of target nucleic acid molecules, the method comprising:
(a) providing a pool of oligonucleotide sets used to detect each of the plurality of target nucleic acid molecules for each of the plurality of target nucleic acid molecules; wherein the oligonucleotide sets each comprises one or more oligonucleotides,
(b) providing, as a first reference combination, a combination of oligonucleotide sets combined from the pool of oligonucleotide sets provided for each of the plurality of target nucleic acid molecules, and checking whether a dimer is formed between oligonucleotide sets of the combination;
(c) replacing an oligonucleotide set with dimer formation in the first reference combination with another oligonucleotide set belonging to the same pool of oligonucleotide sets to provide a combination of oligonucleotide sets, which is different from the first reference combination only in the replaced another oligonucleotide set, and checking whether a dimer is formed between oligonucleotide sets of the combination and whether dimer formation is reduced compared with the first reference combination;
(d) in the combination checking for whether the dimer formation is reduced, providing, as a second reference combination, a combination of oligonucleotide sets with a reduction in dimer formation compared with the first reference combination;
(e) replacing an oligonucleotide set with dimer formation in the second reference combination with another oligonucleotide set belonging to the same pool of oligonucleotide sets to provide a combination of oligonucleotide sets, which is different from the second reference combination only in the replaced another oligonucleotide set, and checking whether a dimer is formed between oligonucleotide sets of the combination; and
(f) in the combination checking for whether the dimer is formed, providing a combination of oligonucleotide sets with no dimer formation; wherein the combination of oligonucleotide sets with no dimer formation is used to simultaneously detect the plurality of target nucleic acid molecules.
